(19) United States
(12) Patent Application Publication
(10) Pub. No.: US 2009/0062309 A1
(43) Pub. Date: Mar. 5, 2009

(54) Therapeutic compositions for the treatment of cardiovascular diseases and methods for use therefor

(76) Inventor: Antonio Delgado-Almeida, Valencia (VE)

Correspondence Address: HARNESS, DICKEY & PIERCE, P.L.C., P.O. BOX 828, BLOOMFIELD HILLS, MI, 48303, US

(21) Appl. No.: 12/231,076

(22) Filed: Aug. 28, 2008

Related U.S. Application Data
(60) Provisional application No. 60/966,383, filed on Aug. 28, 2007.

(51) Int. Cl. A61K 31/4965

(52) U.S. Cl. 514/255.06

(57) ABSTRACT

A pharmaceutical composition comprising an effective amount of at least one (a) an effective amount of at least one 3,5-diamino-6-chloro-N-(diaminomethylene) pyrazinecarboxamide monohydrochloride, dihydrate derivative comprising pirazinoylguanidine, benzamil, dichlorobenzamil, 5-(N,N-dimethyl)-Amiloride, 5-(N-ethyl-N-isopropyl)-Amiloride, (N,N-hexamethylene)-Amiloride, 5-(N-methyl-N-isobutyl)-Amiloride, and Amiloride citrate; (b) an effective amount of a calcium increasing agent; and (c) a pharmaceutically acceptable excipient. Methods are provided for treating a cardiovascular disease (CVD) in a patient diagnosed as having CVD or at risk for developing CVD, the method comprising administering to a subject in need of such treatment a therapeutically effective amount of at least one of 3,5-diamino-6-chloro-N-(diaminomethylene) pyrazinecarboxamide monohydrochloride, dihydrate and/or a derivative thereof.

### CROSS-REFERENCE TO RELATED APPLICATIONS

This application claims the benefit of U.S. Provisional Application No. 60/966,383 filed on Aug. 28, 2007. The entire disclosure of the above application is incorporated herein by reference.

### FIELD

The present disclosure relates to pharmaceutical compositions effective in treating cardiovascular disease in particular coronary heart disease and methods for treating mammalian subjects having coronary heart disease (CHD), diabetes and hypertension.

### BACKGROUND

This section provides background information related to the present disclosure which is not necessarily prior art.

Treatment of Cardiovascular Disease (CVD) is a major health care problem across the entire word, and particularly in the United States. In fact, these life-threatening disorders are a major cause of emergency medical care and hospitalization in the United States, and according the National Center for Health Statistics (NCHS) there were approximately 1,565,000 hospitalizations for primary or secondary diagnosis of an acute coronary syndrome (ACS), 669,000 for unstable angina (UA) and 896,000 for myocardial infarction (MI). In the 2003, NCHS reported 4,497,000 visits to emergency departments for primary diagnosis of CVD, wherein the average age of a person having a first heart attack is calculated at 65.8 years for men and 70.4 years for women.

Further studies provided by the Heart Disease and Stroke Statistics—2007 Update, of the American Heart Association, reported an estimated 79,400,000 American adults (1 in 3) have 1 or more types of CVD. Of these, and 37,500,000 are estimated to be age 65 or older. As a separate diagnosis, high blood pressure or hypertension, accounts for approximately 72,000 000 of subjects (defined as systolic pressure 140 mm Hg or greater and/or diastolic pressure 90 mm Hg or greater, taking antihypertensive medication), Coronary Heart Disease (CHD) incidence approaches 15,800,000 subjects, myocardial infarction for approximately 7,900,000 subjects, and angina pectoris (chest pain secondary to ischemic heart disease) incidence in approximately 8,900,000 subjects.

Angina (chest pain secondary to ischemic heart disease) is one of the most common and an early symptom of coronary heart disease, has been recognized as far back as 1880. Angina still represents a medically unresolved problem. Indeed, treatment of angina in particular, as well as associated conditions as ACS, UA, and MI, typically requires a large number of life-style change recommendations, in addition to dietetic advice, drugs, coronary artery intervention, or coronary bypass surgery aimed to improve symptoms, improve the quality of life of subjects, and even primary or secondary prevention of the CHD. Unfortunately, despite a century of medical advances in pharmaceutical drugs, diagnostic methods and epidemiological studies, the current approach to CVD, and CHD remain complex, and at times frustrating.

Among some of the proposals to combat CHD, a mainstay treatment includes the single “polypill” (aspirin+statin+3 blood pressure lowering agents in half dose, and folic acid) as a strategy to reduce CHD by more than 80%. However, such aims remain presently unresolved and the regression of coronary atherosclerosis using simvastatin and intravascular ultrasound study was determined to be unpractical. It remains to be determined whether these changes will translate to meaningful reductions in clinical events, or whether new anti-thrombotic agents for these CHD subjects can provide an adequate solution. However, the overarching determination in view of the purported successes to date remains whether these results in highly selected patient populations can be matched to the real-world treatment of acute coronary syndromes, which involves both macrovascular CHD and microvascular CHD.

In a recent study called the COURAGE Trial (Clinical Outcomes Utilizing Revascularization and Aggressive Drug Evaluation) conducted in 50 hospital centers across the United States and Canada, results showed that optimal drug treatment with percutaneous coronary interventions (PCI) for stable coronary heart disease, was not more effective than optimal medical therapy alone for preventing cardiovascular events, hospitalization or death, suggesting that drugs, surgical procedures or both were not a statistically effective solution for CHD.

For more than a century, hemodynamic mechanisms involving the coupled tissue O₂/CO₂ gas exchange and H⁺/K⁺ ions transport by hemoglobin (Hb) in red blood cells (RBC) have been well known to the scientific community, and has been termed the so-called Bohr/Haldane Effect. Data to date has been shown that the RBC has a number of critical roles in maintaining normal vascular function, blood flow, tissue oxygenation and acid-base regulation. These critical roles, including the nitric oxygen (NO) transport, NO synthetase expression, platelet aggregation, vascular rheology, and endothelial function, have been the subject of extensive studies by many investigators. Unfortunately, and despite such multiple integrated functions to maintain tissue oxygenation in health and diseases states, the role of RBC has never been of interest in the therapeutic approach of subjects with CHD, specially the ischemic condition of ACS, UA or MI.

### SUMMARY

The present disclosure provides for pharmaceutical compositions comprising Amiloride, or an Amiloride derivative and a calcium increasing agent, in an amount effective to provide at least one of an improvement and elimination of symptoms or morbidity associated with macrovascular CHD, and improvement and elimination of symptoms or associated with microvascular CHD.

In further aspects of the present disclosure, the method comprises treating a cardiovascular disease (CVD), comprising administering a therapeutically effective composition comprising at least one of Amiloride, an Amiloride derivative and a calcium increasing agent to a subject in need thereof.

In a further aspect of the present disclosure methods are provided for treating a cardiovascular disease (CVD) in a patient diagnosed as having CVD or at risk for developing CVD, the method comprises administering to a subject in need of such treatment a therapeutically effective amount of at least one of 3,5-diamino-6-chloro-N-(diaminomethylene) pyrazinecarboxamide monohydrochloride, dihydrate and a derivative thereof, wherein the CVD is associated with a dysfunction in red blood cell H⁺/K⁺ transport, the cardiovascular disease comprises myocardial ischemia, oxidative stress, microvascular coronary heart disease, non-obstructive heart disease, coronary endothelial dysfunction, left ventricular hypertrophy, ventricular arrhythmia, angina, dyspnea, peripheral vascular disease, cerebrovascular disease, stroke, hypertension, and diabetes.

In still further aspects of the present disclosure, methods for the prevention, treatment, and amelioration of symptoms of microvascular CHD, characterized by coronary artery obstruction less than 50%, or occlusion, and methods for the prevention, treatment, and amelioration of symptoms of microvascular CHD, characterized by endothelial dysfunction of coronary arteries, hypertension, vascular diseases, stroke and peripheral artery disease or diabetes comprises administering 3,5-diamino-6-chloro-N-(diaminomethylene) pyrazinecarboxamide monohydrochloride, dihydrate and/or a derivative thereof alone to a CHD patient if the patient's plasma free ionized calcium level is above 1.0 mmol/L

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a photomicrograph of Electron microscopy view of the myocardial capillary bed, and a graphic for ion H⁺/K⁺ exchange coupled by O₂/CO₂ transport by hemoglobin that may have a role in the mechanisms of oxygen transport in subjects with abnormal red blood cell K transport and CHD.

FIG. 2 depicts a coronary angiogram showing multiple coronary obstructions in a left anterior descending artery in a normotensive male (52 yr) as an exemplary symptom of CHD.

FIG. 3A depicts an electrocardiogram (ECG) of a normotensive male (65 y), with history of coronary bypass surgery and myocardial infarction, who developed instable angina 2 years after the infarction. The ECG shows prolonged QRS duration (0.13 msec.), and prolonged QTc segment suggesting intraventricular conduction defect, and non-symmetrical inverted T-waves.

FIG. 3B depicts an ECG trace taken from a patient showing rapid improvement of instable angina after 5 days period of hospitalization. The ECG shows improved T-waves only.

FIG. 3C depicts an ECG trace showing the reversion of T-wave alterations following 6-months of treatment with Amiloride and calcium. No changes occurred in QRS and QTc interval.

FIG. 4A depicts an ECG trace of a female (52 y) subject with a history of hypertension and recent a myocardial infarction and heart failure (NYHA II). The ECG shows bradycardia, and first-degree A/V block, as depicted by a prolonged QRS duration, rectified ST-segment in V4, depressed ST-segment in V5, and elevated ST-segment in V6, with progressive T-wave inversion from V4 to V6.

FIG. 4B depicts an ECG trace taken of the subject in FIG. 4A, at the time of clinical improvement of angina (3 weeks), heart failure and hypertension, 2 months after the addition of Amiloride and calcium gluconolactate to the subject's medication. The ECG shows bradycardia, prolonged QRS duration, and marked QTc prolongation, despite clinical improvement suggesting advanced electrolyte alteration (i.e., hypokalemia, low ionized calcium), while T-waves were improved.

FIG. 5A depicts an ECG trace of a female (43 y) subject with a history of hypertension and left ventricular hypertrophy (LVH) by echocardiography. The ECG trace shows tachycardia, prolonged QRS duration, marked QTc prolongation, and inverted T-waves.

FIG. 5B depicts an ECG trace of the subject in FIG. 5A at the time of reversion of clinical symptoms of exertional angina, and palpitations after 3-months of treatment with Amiloride, antihypertensive and vasodilators drugs. Although the QRS duration on the ECG trace and T-wave voltages improved, the QTc interval remained prolonged.

FIG. 6A depicts an ECG trace of a female (52 y) subject with a history of hypertension and post-infarction angina. The ECG trace shows the subject having sinus tachycardia, prolonged slurred QRS complex, QTc prolongation, and inverted T-waves.

FIG. 6B depicts an ECG trace of the subject in FIG. 6A after rapid reversion of angina (7 days), and clinical control of hypertension. The ECG trace shows a prolonged QRS duration, prolonged QTc, and reversion of T-wave inversion after 5-months of Amiloride treatment.

FIG. 7A depicts a series of ECG traces from a subject, a male (57 yr) subject with a history of angina, diabetes and hypertension (January 2005). The ECG trace shows inverted T-waves (V4-V6, Lead II). After 4 months of Amiloride treatment the angina reversed, while hypertension and diabetes were improved. The ECG trace taken 4 months after shows mild positive T-wave in V4-V5, and low voltage in V6, Lead II (May 2005).

FIG. 7B depicts a series of ECG traces for a subject, a male (59 yr) normotensive, diabetic patient (September 2004). The ECG traces show inverted T-waves (V4-V6, Lead II). After 5 months of Amiloride and calcium treatment, angina reversed, and diabetes improved. The subsequent ECG trace shows normal T-waves (V4, V5, Lead II), and low voltage T-wave in V6 (February, 2006).

FIG. 8 depicts an example of coronary angiography in a white male (52 yr) who is normotensive with 3-vessel diseases in right coronary arteries who was enrolled in a single-blind 2-year Amiloride trial (See Example 7 infra). Areas of arterial obstruction are indicated with white arrows

FIG. 9 depicts an ECG trace of a subject having myocardial necrosis and ischemia as shown in precordial leads V1-V3 (left) and V4-V6 (right). The subject, a white male (74 yr) with extensive infarction, angina and heart failure (2005), with some of the ECG alterations shown with arrows across all precordial leads (V1-V6).

FIG. 10A depicts an ECG trace of a male (69 yr) with extensive AMI, emergency angioplasty, and LAD stent (2006), with sustained ECG changes shown with arrows. The ECG was taken 1-year after the AMI (2007).

FIG. 10B depicts an ECG trace of the subject in FIG. 10A illustrating persistent ECG alterations as indicated with arrows despite receiving optimal medical treatment. The subject did not present with angina and had a Duke treadmill Score of −1. (February 2008). This subject is a control, non-treated subject in the single-blind 2-year Amiloride trial (See Example 7 infra).

FIG. 11A depicts an ECG trace from a white male (74 yr) depicted in FIG. 9 with previous extensive myocardial infarction, angina and heart failure with identical ECG alterations 1-year later (2006).

FIG. 11B depicts an ECG trace of the subject in FIG. 11A, depicting an unchanged ECG during a 3-year follow-up, despite receiving optimal medical treatment. The subject had angina and presented with a Duke Treadmill Score of −3. Results obtained before entering the single-blind 2-year Amiloride trial (See Example 7 infra) in (January 2008).

FIG. 11C depicts an ECG trace of the subject in FIG. 11B, showing a rapid reversion of ST-T alterations (V2-V6) 2-months after Amiloride and calcium treatment. The subject continued with his other medications as taken in FIG. 11B. The subject was free of angina and symptoms of heart failure, with a Duke Treadmill Score of +4.3 (2008).

FIG. 12A depicts an ECG trace from a White male (65 yr) taken 7 months after experiencing an extensive AMI, arrhythmia, and heart failure. Although he was free of angina or symptoms of heart failure during the Amiloride trial, oral calcium and optimal medical treatment, the ECG still had marked ischemic changes (ST-segment from V1-V4, inverted T-wave in V1-V5).

FIG. 12B depicts an ECG trace of the subject in FIG. 12A showing significant ECG improvement of ST-segment, and T-waves (V1-V6), 1-year post the infarction. The subject was enrolled in the single-blind 2-year Amiloride trial (See Example 7 infra). The ECG was taken 5 months after commencing treatment with Amiloride, calcium and statins.

FIG. 12C depicts an ECG trace taken from the subject in FIG. 12B showing essentially a normal ECG trace 18 months post AMI. The subject was asymptomatic and received Amiloride, calcium and statins and no other medication. The subject's Duke Treadmill Scores improved to 5.49 and 6.23. Normal left ventricular contractility and function in serial echocardiograms were observed in 2006-2007.

FIG. 13A depicts an ECG trace from a Hispanic male (54 yr) subject presenting with diabetes, angina, and a Duke Treadmill Score of −3. The subject was being treated with an optimal anti-anginal and diabetes treatment (2004).

FIG. 13B depicts an ECG trace showing improvement of ischemic changes as shown in the ECG trace at (V3-V6) following 5-months of Amiloride and statins, regular treatment for glycemic control but no other medication. Improved DT Score=2.3 (2005)

FIG. 13C depicts recurrence of ECG alterations after 6-months without Amiloride as shown by the arrows, despite treating the subject with optimal angina and diabetes medications. Three months after Amiloride was reinitiated, the subject's ECG results were shown to be normal without ischemic changes (ECG not shown)

FIG. 14A depicts ECG tracings from a Black diabetic female (69 yr) having had an inferior AMI. The ECG trace reflects improvement in the subject's antero-lateral ischemia indicated with arrows during admission in the Amiloride trial, having normal precordial leads.

FIG. 14B depicts an ECG trace taken of the subject in FIG. 14A two months after withdrawal of Amiloride treatment. The ECG shows ECG alterations marked with arrows indicative of acute coronary syndrome and ventricular arrhythmias. This subject was admitted to hospital.

### DETAILED DESCRIPTION

The following description is merely exemplary in nature and is not intended to limit the present disclosure, application, or uses.

The pharmaceutical compositions of the present disclosure include an Amiloride derivative and a calcium increasing agent. The amount of the Amiloride derivative and the calcium increasing agent is effective in the treatment and/or prevention of CVD, or reduction or improvement of one or more CVD symptoms related to dysfunction in red blood cell H⁺/K⁺ transport. The two active agents Amiloride and/or an Amiloride derivative and a calcium increasing agent, can be co-administered in a single formulation or in separate formulations and the active agents can be provided one or more times per day to reduce the mortality and morbidity due to or associated with CVD.

### Amiloride

Amiloride particularly in its hydrochloride salt form is specifically contemplated as having utility in the present disclosure. Amiloride is an antikaliuretic-diuretic agent and is chemically identified as a 3,5-diamino-6-chloro-N-(diaminomethylene) pyrazinecarboxamide monohydrochloride, dihydrate and has a molecular weight of 302.12. Amiloride has a designated empirical formula of C₆H₈ClN₇O—HCl.2H₂O and has a structural formula:

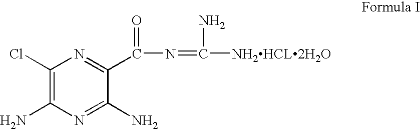

In some embodiments, pharmaceutical compositions of the present disclosure can include Amiloride and/or can include one or more Amiloride derivatives including, for example, pyrazinoylguanidine, benzamil, dichlorobenzamil, 5-(N,N-dimethyl)-Amiloride, 5-(N-ethyl-N-isopropyl)-Amiloride, (N,N-hexamethylene)-Amiloride, 5-(N-methyl-N-isobutyl)-Amiloride, and Amiloride citrate.

Upon ingestion, Amiloride is pharmacologically unchanged when metabolized, particularly in humans. Amiloride is not metabolized by the liver and is excreted from the kidneys unchanged

In some embodiments, references to Amiloride of formula (I) include references to salts, solvates, multi-component complexes and liquid crystals thereof and to solvates, multi-component complexes and liquid crystals of salts thereof.

The compounds of the present disclosure can include compounds of formula (I) as defined above, including all polymorphs thereof, (including optical, geometric and tautomeric isomers) as hereinafter defined and isotopically-labeled compounds of formula (I).

In some embodiments, so-called ‘prodrugs’ of the compounds of formula (I) are also within the scope of the present disclosure. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the present disclosure can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985). Examples of replacement groups in accordance with the foregoing prodrug types can be found in the aforementioned references.

In some embodiments, stereoisomers, geometric isomers and tautomeric forms of Amiloride and/or Amiloride derivatives, analogs and the like, including compounds exhibiting more than one type of isomerism, and mixtures of one or more thereof are also contemplated. Also included are acid addition or base salts wherein the counterion is optically active. Cis/trans isomers may be separated by conventional techniques well known to those skilled in the art, for example, chromatography and fractional crystallization.

Chiral compounds of Amiloride (and chiral precursors thereof) may be obtained in enantiomerically enriched form using chromatography, typically HPLC, on an asymmetric resin with a mobile phase consisting of a hydrocarbon, typically heptane or hexane. Racemic mixtures may be separated by conventional techniques known to those skilled in the arts. See, for example, Stereochemistry of Organic Compounds by E. L. Eliel and S. H. Wilen (Wiley, 1994).

Amiloride for use in the present therapeutic pharmaceutical compositions is commercially available from Jai Radhe Sales, Ahmedabad-380, Gujrat, INDIA.

The amount of an Amiloride active, i.e. one or more Amiloride actives combined, the Amiloride active includes for example, Amiloride, an Amiloride derivative, a pharmaceutically acceptable salt thereof, or a pharmaceutically acceptable solvate of any of the foregoing present in a unit dose, (whether the dose is an oral dose or a parenteral liquid dose), can range from about 0.05 mg to about 100 mg, preferably from about 0.1 mg to about 50 mg, and more preferably from about 1 mg to about 25 mg. As used herein, the pharmaceutical compositions provided by the present disclosure can be formulated in a unit dosage form. Unit dosage form refers to a physically discrete unit suitable as a unitary dose for patients undergoing treatment, with each unit containing a predetermined quantity of one or more Amiloride actives calculated to produce an intended therapeutic effect. A unit dosage form can be for a single daily dose or one of multiple daily doses, e.g., 2 to 4 times per day. When multiple daily doses are used, the unit dosage can be the same or different for each dose. One or more dosage forms can comprise a dose, which may be administered to a patient at a single point in time or during a time interval.

### Calcium Increasing Agent

The compositions of the present disclosure, includes as part of a combination treatment, one or more calcium increasing agents. A calcium increasing agent can be any source of calcium that is capable of raising the free ionized calcium concentration in the plasma to at least 1.0 mmol/L (2.0 mEq/L) in the plasma of a patient with CHD. Calcium (Ca²⁺) must be present in a soluble form for efficient absorption from the gastrointestinal tract. This soluble form is generally Ca²⁺ ions, which are hydrated by solvent water or loosely complexed to anionic ligands in solution. Calcium absorption from gastrointestinal tract takes place by both active transport and passive diffusion. The active transport is saturable, stimulated by 1,25-dihydroxyvitamin D3, and predominates in the duodenum and proximal jejunum; while the passive transport is the principal mechanism for absorbing large loads of Ca²⁺ (which saturate the active process), and involves the distal jejunum and ileum which have longer transit times.

Many of the studies of Ca²⁺ absorption have employed a single oral dose, ranging from 250 mg up to 2000 mg. With a smaller dose, 15-500 mg of Ca²⁺, the fractional absorption varies inversely with the logarithm of the load size (Heaney R P, Weaver C M, and Fitzsimmons M L (1990) Influence of calcium load on absorption fraction. J Bone Miner Res 5: 1135-1138). With a larger load (>500 up to 2000 mg), the fractional absorption increases linearly with load size, reflecting a role of the passive transport over the active transport.

Calcium absorption has also been modeled by fitting the amount absorbed to an equation with a hyperbolic term for the saturable process and a linear term for the nonsaturable process (Blanchard J and Aeschlimann J-M (1989) Calcium absorption in man: some dosing recommendations. J Pharmacokinet Biopharm 17: 631-644).

In various embodiments of the present disclosure, the calcium increasing agent can include elemental calcium, calcium salt and complexed calcium supplements. In various embodiments, the calcium increasing agent is a calcium salt. In some embodiments, the calcium increasing agent can be elemental calcium or a calcium salt or complexed calcium that upon a single and/or multiple administration doses over a period of hours to days, days to weeks, or weeks to months, raises the plasma concentration of free ionized calcium to at least 1.0 mmol/L (2.0 mEq/L) in the subject.

Free ionized calcium is amenable to calculation once the concentration of total plasma calcium concentration is known. The total plasma calcium concentration consists of three fractions. Approximately 15 percent is bound to multiple organic and inorganic anions such as sulfate, phosphate, lactate, and citrate. About 40 percent is bound to albumin in a ratio of 0.8 mg/DI (0.2 mmol/L or 0.4 mEq/L) of calcium per 1.0 g/DI (10 g/L) of albumin. The remaining 45 percent circulates as physiologically active ionized (or free) calcium. The ionized calcium concentration is tightly regulated by parathyroid hormone and vitamin D. The wide range in the normal total plasma calcium concentration is probably due to variations in the plasma concentration of albumin among normal healthy individuals and to variations in the state of hydration and arterial acid-base balance or increased blood concentrations of lactate, citrate and the like in subjects that can alter the albumin concentration and thus the free ionized calcium levels. The net effect is that measurement of the total plasma calcium concentration alone can be misleading, since these parameters can change affecting the ionized calcium fraction. For a more detailed explanation of free ionized calcium measurement, corrections and discussion please see Dickerson, R. N. et al., J. Parenteral and Enteral Nutrition. (2004) 28(3):133-141, and Moore, E. W. (1970). J. Clin. Invest. 49(2): 318-334.

In some embodiments, the calcium increasing agent can include calcium supplements. The compositions of the present disclosure can also be formulated with one or more calcium supplements as described in Table 1 below, by providing sufficient calcium supplement in the composition to reach a predetermined elemental calcium amount for example, from about 101 mg to about 5000 mg, from 100 mg to about 2000 mg, from about 500 mg to about 1,000 mg in the composition. Table 1 can be used to calculate the amount of calcium supplement required to reach a predetermined elemental calcium amount.

Table 1. Relationship between free calcium, supplement strength and number of calcium tablets required to achieve a desired amount of 1000 mg of elemental calcium in commonly used and available forms of supplemental calcium. In the present investigation, the preferable calcium composition to increase ionized calcium above 1.0 mmol/L were: Calcium Sandoz (7.2 g of calcium salts) providing 1-g of free calcium, and Ideos Calcium (1.25 g of calcium salts) providing about 300 mg of free calcium. For other calcium preparations the measurement of ionized calcium after “in vitro” tablet disintegration, along with the measurement of plasma ionized calcium in the studied subjects is warranted,

TABLE 1

| | | | Number of |
|---|---|---|---|
| | Strength of | Amount of elemental | tablets to |
| | each tablet | calcium | provide 1000 |
| Calcium | (in | per tablet | milligrams of |
| supplement | milligrams) | (in milligrams) | elemental calcium |
| Calcium | 625 | 250 | 4 |
| carbonate | | | |
| | 650 | 260 | 4 |
| | 750 | 300 | 4 |
| | 835 | 334 | 3 |
| Calcium Ideos | 1250 | 500 | 2 |
| | 1500 | 600 | 2 |
| Calcium citrate | 950 | 200 | 5 |
| Calcium | 500 | 45 | 22 |
| gluconate | | | |
| | 650 | 58S | 17 |
| | 1000 | 90 | 11 |
| Calcium | 325 | 42 | 24 |
| lactate | | | |
| | 650 | 84 | 12 |
| Calcium | 500 | 115 | 9 |
| phosphate, | | | |
| dibasic | | | |
| Calcium | 800 | 304 | 4 |
| phosphate, | | | |
| tribasic | | | |
| | 1600 | 608 | 2 |
| Calcium | | 1000 | 1 |
| Sandoz | | | |
| Calcium | 5230 | | |
| gluconolactate | | | |
| Calcium | 2300 | | |
| carbonate | | | |
| GramCal | | 1000 | 1 |
| | | (50 mEq) | |
| Calcium | 2327 | | |
| lactate | | | |
| Calcium | 1750 | | |
| Gluconate | | | |
| Calcium | | 500 | 2 |
| Sandoz Forte | | | |

In some embodiments, the amount of calcium increasing agent to be included in the composition, shall be an effective amount of calcium increasing agent that is capable of raising the plasma free ionized calcium concentration to at least 1.0 mmol/L. The typical amount of the calcium increasing agent to be incorporated into the compositions of the present disclosure depends on several factors that are readily calculated and determined by one of ordinary skill in the art, for example, the amount of elemental calcium in the supplement, the amount of ionized calcium after in vitro tablet disintegration, and patient's plasma level of free ionized calcium prior to treatment or administration of the compositions, the age of the subject, the sex of the subject, the presence of medications or supplements with calcium depleting effects among others.

### Administration

The active agents of the present disclosure intended for pharmaceutical use may be administered as crystalline, liquid or amorphous products. They may be obtained, for example, as solid plugs, powders, or films by methods such as precipitation, crystallization, freeze drying, spray drying, or evaporative drying.

In some embodiments of the methods of the present disclosure, a composition comprising Amiloride may be administered alone or in combination with a calcium increasing agent and optionally in combination with one or more other drugs, particularly drugs for treatment angina (i.e., nitrates, aspirin, folic acid, and other established drugs for CHD), hypertension, diabetes or hyperlipidemia, and or bioactive substances (or as any combination thereof). Generally, the Amiloride and/or calcium increasing agent can be administered as a formulation in association with one or more pharmaceutically acceptable excipients. The term ‘excipient’ is used herein to describe any ingredient other than the compound(s) of the present disclosure. The choice of excipient will to a large extent depend on factors such as the particular mode of administration, the effect of the excipient on solubility and stability, and the nature of the dosage form.

In some embodiments, the pharmaceutical composition comprising Amiloride and/or an Amiloride derivative is designed to have no additive color, and preferably use the excipients lactose, starch, sugar, gelatin powder, and magnesium stearate.

Pharmaceutical compositions suitable for the delivery of calcium-increasing agent and methods for their preparation will be readily apparent to those skilled in the art. Such compositions and methods for their preparation may be found, for example, in Remington's Pharmaceutical Sciences, 19th Edition (Mack Publishing Company, 1995).

The compounds of the disclosure may be administered orally. Oral administration may involve swallowing, so that the compound enters the gastrointestinal tract, and/or buccolingual, or sublingual administration of Amiloride by which this drug enters the blood stream directly from the mouth. Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

For tablet dosage forms, depending on quantity of the active agent or active agents, the active agent or active agents may make up from 0.0001 weight % to 99 weight % of the dosage form, more typically from 0.001 weight % to 90 weight % of the dosage form. In addition to the active agent(s), tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate; zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colorants, flavoring agents, preservatives and taste-masking agents.

Exemplary tablets contain up to about 80% of the active agents, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula I, a film-forming polymer, a binder, a solvent, a humectant, a plasticiser, a stabiliser or emulsifier, a viscosity-modifying agent and a solvent. Some components of the formulation may perform more than one function.

Other possible additives can include anti-oxidants, colorants, flavorings and flavor enhancers, preservatives, salivary stimulating agents, cooling agents, co-solvents (including oils), emollients, bulking agents, anti-foaming agents, surfactants and taste-masking agents, effervescent inducing agents that cause the composition to fizz or effervesce when added to an aqueous solution, for example, carbonates, bicarbonates and the like.

Solid formulations for oral administration may be formulated to be immediate, for example, as in sublingual preparations of Amiloride, and/or modified release. Modified release formulations include delayed-, sustained-, pulsed-, controlled-, targeted and programmed release.

The compounds of the disclosure may also be administered directly into the blood stream, into muscle, or into an internal organ. Injectable compositions can be pharmaceutical compositions for any route of injectable administration, including, but not limited to, intravenous, intraarterial, intracoronary, pericardial, perivascular, intramuscular, subcutaneous, intradermal, intraperitoneal, and intraarticular. In certain embodiments, an injectable pharmaceutical composition can be a pharmaceutically appropriate composition for administration directly into the heart, pericardium or coronary arteries. Suitable devices for parenteral administration include needle (including microneedle) injectors, needle-free injectors, infusion techniques and transdermal patch delivery technologies commonly known in the pharmaceutical arts.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile water or physiological saline. The preparation of parenteral formulations under sterile conditions, for example, by lyophilization, may readily be accomplished using standard pharmaceutical techniques well known to those skilled in the art.

The solubility of either Amiloride, calcium increasing agent, or both used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the disclosure may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(dl-lactic-coglycolic)acid (PGLA) microspheres.

The compounds of the disclosure may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated. Other means of topical administration include delivery by electroporation, iontophoresis, phonophoresis, sonophoresis and microneedle or needle-free (e.g. Powderject™, Bioject™ etc.) injection. Formulations for topical administration may be formulated to be immediate and/or modified release. Modified release formulations include delayed-, sustained-, pulsed-, controlled-, targeted and programmed release.

Capsules (made, for example, from gelatin or hydroxypropylmethylcellulose), blisters and cartridges for use in an inhaler or insufflator may be formulated to contain a powder mix of the Amiloride and/or an Amiloride derivative and optionally a calcium increasing agent of the disclosure, a suitable powder base such as lactose or starch and a performance modifier such as l-leucine, mannitol, or magnesium stearate. The lactose may be anhydrous or in the form of the monohydrate. Other suitably known sugar excipients can include dextran, glucose, maltose, sorbitol, xylitol, fructose, and sucrose.

The two active agents of the present disclosure can be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

### Kits

Pharmaceutical compositions comprising the two active agents can be administered, for example, for the purpose of treating a particular cardiovascular disease or condition. It is within the scope of the present disclosure that at least one of the active agents can be formulated in one or more dosage forms such that at least one of the active agents may be conveniently combined in the form of a kit suitable for co-administration of the active agents and/or dosage forms.

In some embodiments, the kits of the present disclosure comprises one or more separate pharmaceutical compositions, at least one of which contains Amiloride, and/or an Amiloride derivative and at least one of which contains a calcium increasing agent. Preferably, the kit contains one composition comprising both active agents formulated into one dosage form, in accordance with the present disclosure. In some embodiments the kit also contains a means for separately retaining the active agents, such as a container, divided bottle, or divided foil packet. An example of such a kit is the familiar blister pack used for the packaging of tablets, capsules and the like.

In some embodiments, the kits of the present disclosure are particularly suitable for administering different dosage forms, for example, oral and parenteral, for administering the at least one or both active agents at different dosage intervals, or for titrating the separate active agents against one another. To improve patient compliance, the kit typically comprises directions for administration and can optionally be provided with a so-called memory aid.

### Methods of Treatment

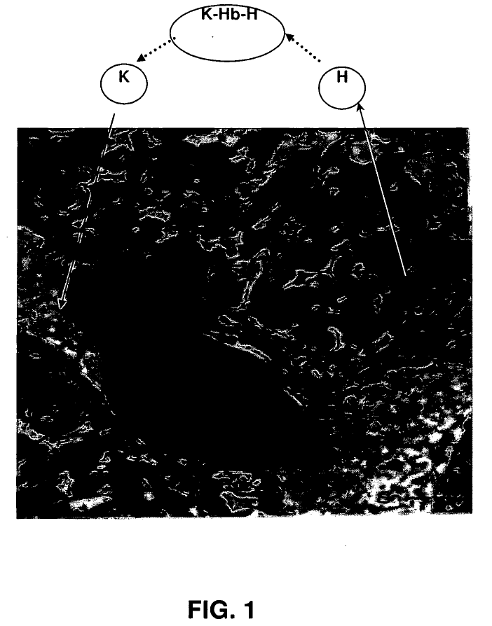

In some embodiments of the present disclosure, pharmaceutical compositions comprising Amiloride and/or Amiloride derivative and a calcium increasing agent can be utilized in methods for treating subjects with Cardiovascular Diseases, for example, CHD, left ventricular hypertrophy, hypertension, and diabetes, and in particular, cardiovascular disease subjects having a plasma free ionized calcium concentration of less than 1.0 mmol/L. CHD can be summarily described as a constriction (stenosis) of at least one blood supplying artery to the heart, the so-called macrovascular disease, and the impaired coronary blood supply in absence of mayor coronary artery obstruction, the so-called microvascular disease; the former more common in men, the later in_women who may have acute myocardial infarction (AMI) despite normal coronary arteries. Often, the result of a coronary occlusion results in deprivation of oxygen to the cardiac tissue (ischemia) or the prevention of induced blood flow to the occluded artery. In some CHD subjects, the occlusion can arise as a result of atherosclerotic plaque and/or calcified coronary arteries, adjacent and proximate to the arterial walls, thus impeding the flow of oxygen and nutrients to the heart. In cases with microvascular coronary heart disease, the impaired blood supply results from distinctive pathogenic mechanisms, i.e., an increased left ventricular mass in LVH, endothelial dysfunction in diabetes mellitus, increased red blood cell and platelet aggregation in dyslipidemia and particularly, a hereditable or acquired defect in red blood cell potassium uptake, and cellular potassium transport that may disrupt the physiological mechanisms of coupled H⁺/K⁺ and O₂/CO₂ exchanges at the myocardium capillary bed illustrated in FIG. 1.

In some embodiments, the active agents, Amiloride and/or an Amiloride derivative and a calcium increasing agent can be co-administered in a single dosage form, separate dosage forms, or are administered sequentially. “Co-administration” of a combination of Amiloride (and/or its salts, and derivatives) and one or more calcium increasing agents can include administration of Amiloride and/or Amiloride derivative and one or more calcium increasing agent in a unitary formulated dosage form, for example, as a tablet, as a powdered dose, or a liquid solution or suspension. “Co-administration” also includes administering Amiloride and/or an Amiloride derivative and a calcium increasing agent separately or sequentially but as part of the same therapeutic treatment program or regimen. The components need not necessarily be administered at essentially the same time, although they can if so desired. Thus “co-administration” includes, for example, administering Amiloride and/or Amiloride derivative and a calcium increasing agent as separate dosages or dosage forms, but at the same time. “Co-administration” also includes separate administration at different times and in any order. For example, where appropriate a patient may take one or both active agent(s) of the treatment in the morning and the other or both active agent(s) at night.

In some embodiments, the methods of the present disclosure consist of administering Amiloride and/or an Amiloride derivative as the single active agent to subjects having a cardiovascular disease, including, for example, CHD, in an effective amount to reduce one or more indicators of macrovascular CHD as in subjects with coronary obstruction. In some embodiments, the CHD can also include microvascular CHD in subjects with coronary endothelial dysfunction, left ventricular hypertrophy, hypertension, or diabetes. In some embodiments of the methods described in the present disclosure, a subject having CVD can be therapeutically or prophylactically administered with at least one of Amiloride, an Amiloride derivative and a calcium increasing agent, each in an amount that substantially improves or reverses at least one clinical symptom related to CVD selected from the group consisting of: improvement or normalization of an ST-segment alteration, T-wave alteration, improvement or normalization of voltage of LVH, and/or LV strain in LVH, improved Duke Treadmill Score, a reduction in the severity or duration of angina pectoris, exertional dyspnea or arrhythmias, improved LV contractility, a normalization of red blood-cell potassium homeostasis and a reduction in frequency or duration of a ventricular arrhythmias. Administration of Amiloride and/or an Amiloride derivative alone can be particularly indicated for subjects having a plasma ionized free calcium concentration of 1.0 mmol/L or above.

### Subjects

The present disclosure provides for the treatment of cardiovascular diseases. Generally speaking, cardiovascular disease can occur in subjects in which insufficient oxygen exchange is occurring between the red blood cell supplied to the tissue and the tissue for which is deficient in oxygen. The present inventor has discovered that the pharmaceutical compositions of the present disclosure improves and/or ameliorates deficient oxygen transport between the red blood cell and the intended cellular recipient. In effect, the present methods can be applied to cardiovascular diseases in which there is insufficient oxygen exchange between the red blood cells in circulation and the cells or tissue to be oxygenated. In some embodiments, the etiology of the oxygen insufficiency is not due to a macro vascular blockage as seen in coronary artery disease but rather a microvascular disease as described above. Therefore, in some embodiments, the cardiovascular diseases that can be treated with the present pharmaceutical compositions comprising at least one of Amiloride, and an Amiloride derivative; and optionally a calcium increasing agent can include: hypertension (e.g. pulmonary hypertension, labile hypertension, idiopathic hypertension, low-renin hypertension, salt-sensitive hypertension, low-renin, salt-sensitive hypertension, thromboembolic pulmonary hypertension; pregnancy-induced hypertension; renovascular hypertension; hypertension-dependent end-stage renal disease, hypertension associated with cardiovascular surgical procedures, hypertension with left ventricular hypertrophy, and the like), LV diastolic dysfunction, unobstructive coronary heart diseases, myocardial infarctions, cerebral infarctions, peripheral vascular disease, cerebrovascular disease, cerebral ischemia, angina, (including chronic, stable, unstable and variant (Prinzmetal) angina pectoris), aneurysm, ischemic heart disease, myocardial ischemia, thrombosis, platelet aggregation, platelet adhesion, smooth muscle cell proliferation, vascular or non-vascular complications associated with the use of medical devices, wounds associated with the use of medical devices, vascular or non-vascular wall damage, peripheral vascular disease, neointimal hyperplasia following percutaneous transluminal coronary angiography, vascular grafting, coronary artery bypass surgery, thromboembolic events, post-angioplasty rest enosis, coronary plaque inflammation, hypercholesterolemia, embolism, stroke, shock, arrhythmia, atrial fibrillation or atrial flutter, thrombotic occlusion and reclusion cerebrovascular incidents, left ventricular dysfunction and cardiac hypertrophy. More preferably, the cardiovascular disease is hypertension with left ventricular hypertrophy and/or unobstructive CHD.

In some embodiments, subjects for treatment by the methods described herein can be mammalian subjects, preferably human, who have been diagnosed with or at risk of developing a CVD, the CVD includes: myocardial ischemia, oxidative stress, non-obstructive coronary heart disease, microvascular coronary heart disease, coronary endothelial dysfunction, left ventricular hypertrophy, ventricular arrhythmia, angina, dyspnea, peripheral vascular disease, cerebrovascular disease, stroke, hypertension, and diabetes. In some embodiments, the cardiovascular disease is accompanied by symptomatic myocardial ischemia. Non-obstructive coronary heart disease is defined herein as having less than 50% blockage of the coronary arteries or less than 30% of blockage of the coronary arteries. Hence, coronary artery disease (CAD) or obstructive CHD is defined as a blockage of the coronary arteries, wherein the blockage is more than 50% in at least one major artery. Diseases associated with myocardial ischemia can include stable angina, unstable angina, and myocardial infarction. In some embodiments, candidates for the methods described herein will be subjects with stable angina and reversible myocardial ischemia. Stable angina is characterized by constricting chest pain that occurs upon exertion or stress, and is relieved by rest or sublingual nitroglycerin. Coronary angiography of subjects with stable angina usually reveals 50-70% obstruction of at least one coronary artery. Stable angina is usually diagnosed by the evaluation of clinical symptoms and ECG changes. Subjects with stable angina may have transient ST segment abnormalities, but the sensitivity and specificity of these changes associated with stable angina are low.

In some embodiments, candidates for the methods described herein will be subjects, for example, human subjects with unstable angina and reversible myocardial ischemia. Unstable angina is characterized by constricting chest pain at rest that is relieved by sublingual nitroglycerin. Anginal chest pain is usually relieved by sublingual nitroglycerin, and the pain usually subsides within 30 minutes. There are three commonly described classes of unstable angina severity: class I, characterized as new onset, severe, or accelerated angina; class II, subacute angina at rest characterized by increasing severity, duration, or requirement for nitroglycerin; and class III, characterized as acute angina at rest.

Unstable angina represents the clinical state between stable angina and acute myocardial infarction (AMI) and is thought to be primarily due to the progression in the severity and extent of atherosclerosis, coronary artery spasm, or hemorrhage into non-occluding plaques with subsequent thrombotic occlusion. Coronary angiography of subjects with unstable angina usually reveals 90% or greater obstruction of at least one coronary artery, resulting in an inability of oxygen supply to meet even baseline myocardial oxygen demand. Slow growth of stable atherosclerotic plaques or rupture of unstable atherosclerotic plaques with subsequent thrombus formation can cause unstable angina. Both of these causes result in critical narrowing of the coronary artery. Unstable angina is usually associated with atherosclerotic plaque rupture, platelet activation, and thrombus formation. Unstable angina is usually diagnosed by clinical symptoms, ECG changes, and changes in cardiac markers.

In some embodiments, subjects for the methods described herein will be subjects having had or are at risk for having a MI or are undergoing an AMI. MI is characterized by constricting chest pain lasting longer than 30 minutes that can be accompanied by diagnostic ECG Q waves. Most subjects with AMI have CHD, and as many as 25% of AMI cases are “silent” or asymptomatic infarctions. AMI is usually diagnosed by clinical symptoms, ECG changes, and elevations of cardiac proteins, most notably cardiac troponin, creatine kinase-MB and myoglobin.

In some embodiments, candidates for the methods described herein can be human subjects with left ventricular dysfunction and reversible myocardial ischemia. In some embodiments, subjects that can be treated by the compositions of the present disclosure, can include cardiovascular disease subjects as having any one or more of: characterized as having evidence of myocardial ischemia in resting ECG involving abnormalities in the QRS complex (Q-waves from previous MI, decreased R-wave height, non-progressive R-wave voltages in precordial leads), ST-segment (ST-segment elevation ≧0.1 μV often referred as injury current, ST-segment depression ≧0.1 μV often downsloping from the J point, rectification of the ST segment), T-waves voltage or morphology (isoelectric T-wave, low voltage T-wave, biphasic, negative or inverted T-waves, peaked T-wave in acute ischemia), intraventricular conduction disturbances, and arrhythmias (usually as ventricular extrasystoles); characterized as having evidence of “silent ischemia” by continuous ST-segment monitoring in Holter test (asymptomatic ST-segment and T-waves changes, intraventricular disturbances or arrhythmia, as in the resting ECG); characterized as having evidence of myocardial ischemia in pharmacological stress test (intravenous graded infusion of dipyridamole, adenosine, or dobutamine), and assessed by echocardiographic changes in Left Ventricular contractility (abnormal LV motion, areas of hypokinesis, LV diastolic dysfunction); characterized as having evidence of myocardial ischemia in Exercise Stress testing (angina, ST-segment depression, ST-segment elevation, intraventricular conduction disturbances, or arrhythmia), by Bruce Protocol, and quantified by Duke Treadmill Score, or other well-known indexes; characterized as having angina, dyspnea, or arrhythmia related to CHD in normotensive and hypertensive subjects; characterized as having CHD; is hypertensive; is diabetic; has had dyslipidemia; experienced a previous MI; has had an angiography for coronary obstruction; or has had a post coronary bypass surgery, or coronary interventional procedure; has had correction of electrolyte abnormalities in plasma electrolytes; has had omission of drugs that may affect ventricular repolarization, as digoxin, furosemide, β-blocker, or others as such; has had ruled out other non CHD causes of chest pain, dyspnea, and arrhythmia.

In some embodiments, subjects for the methods described herein will be human subjects with left ventricular hypertrophy (LVH) by ECG (ECG-LVH), increased LV mass (as measured using Echocardiogram-LVH), and LV dysfunction. In some embodiments, the subjects that can be treated by the compositions of the present disclosure include cardiovascular disease subjects as having any one or more of: increased left ventricular voltage criteria (Cornell Double-Product, Sokolow-Lyon, Romhilt-Estes, Framingham Heart Study, or Brazilian new score for Left Ventricular Hypertrophy), along with typical LV strain pattern (ST depression opposite to QRS with inverted or biphasic T waves), or LVH voltage criteria with abnormal T-wave voltage or morphology (low voltage T-waves, isoelectric or non-symmetrical negative T-waves) without ST-segment depression; characterized as having angina, exertional dyspnea, or arrhythmia related to CHD in hypertensive subjects; characterized as having CHD; is hypertensive; is diabetic; has had dyslipidemia; experienced a previous myocardial infarction; has had an angiography for coronary obstruction; has had a post coronary bypass surgery, or has had coronary interventional procedure; has had correction of electrolyte abnormalities in plasma electrolytes; has had omission of drugs that may affect ventricular repolarization, as digoxin, furosemide, β-blocker, or others as such; has had ruled out other non CHD causes of chest pain, dyspnea, and arrhythmia.

In some embodiments, subjects contemplated for the methods described herein will be subjects having coronary heart disease but who are also normotensive. As defined herein, normotensive subjects are those subjects with a systolic blood pressure of less than 90 mmHg and a diastolic blood pressure less than 140 mmHg. (Definition in accordance with the American Heart Organization).

In some embodiments, subjects contemplated for the methods described herein will be subjects having cardiovascular disease but who are also normotensive as defined above.

### Dosages

An effective amount of the therapeutic composition, can illustratively include an amount of the therapeutic composition (e.g., a composition comprising an Amiloride and/or an Amiloride derivative or Amiloride and/or an Amiloride deriviative and a calcium increasing agent) sufficient to produce a measurable biological response (e.g., improve and/or reverse the electrocardiographic abnormalities related to the CHD, improves or reverses at least one clinical symptom related to left ventricular hypertrophy or CHD, improves the erythrocyte potassium content without any secondary effects, decreases enteral sodium chloride absorption while decreasing renal sodium chloride and water excretion, improves tolerance to exercise as tested using a Duke Treadmill test and measured by an increase in the Duke Treadmill Score and/or to improve tolerance and/or or minimize risk associated medicaments that are potassium sparing agents, particularly, the absence of clinical or ECG alteration of hyperkalemia, as in our trials with Amiloride.

Actual dosage levels of active agents in a therapeutic pharmaceutical composition of the present disclosure can be varied so as to administer an amount of Amiloride and/or an Amiloride derivative and if the plasma ionized Ca²⁺ level is lower than 1.0 mmol/L, a calcium increasing agent that is effective to achieve the desired therapeutic response i.e. to raise the plasma ionized Ca²⁺ level at or above 1.0 mmol/L for a particular subject and/or application. The selected dosage level will depend upon a variety of factors including the activity of the therapeutic composition, formulation, the route of administration, combination with other drugs or treatments, severity of the condition being treated, and the physical condition and prior medical history of the subject being treated.

Preferably, a minimal dose of Amiloride and/or an Amiloride derivative with or without calcium increasing agent can be administered, and the Amiloride and/or Amiloride derivative dose can be escalated in the absence of dose-limiting toxicity to a minimally effective amount. Determination and adjustment of a therapeutically effective dose, as well as evaluation of when and how to make such adjustments, are known to those of ordinary skill in the art of cardiac medicine.

In some embodiments, the actual amount or quantity of each active agent can be determined on a mg/kg scale in accordance with the known pharmacological activities and estimated residence time of each active in the subject's body. In some-embodiments, body surface area can be used as a common denominator for drug dosage in adults and children as well as in different animal species as described by Freireich et al. (1966) Cancer Chemother Rep 50:219-244. Briefly, to express a mg/kg dose in any given species as the equivalent mg/sq m dose, multiply the dose by the appropriate kg/sq.m factor. As an illustration in an adult human, 100 mg/kg is equivalent to 100 mg/kg×37 kg/sq m=3700 mg/m².

For oral administration, a satisfactory result can be obtained employing a pharmaceutical composition comprising Amiloride and/or an Amiloride derivative in an amount ranging from about 0.001 mg/kg to about 10 mg/kg and preferably from about 0.01 mg/kg to about 2 mg/kg. A preferred oral dosage form, such as tablets or capsules, will contain Amiloride and/or an Amiloride derivative in an amount ranging from about 0.05 to about 100 mg, preferably from about 0.1 to about 50 mg, and more preferably from about 1 to about 25 mg. Exemplary doses for oral administration are also provided in Examples 2 and 3.

Similarly, a satisfactory result can be obtained employing a co-administered amount of a calcium increasing agent with the Amiloride and/or an Amiloride derivative, for example, one or more calcium supplements described in Table 1 herein in an amount which provides upon administration, an amount of elemental calcium sufficient to raise the plasma free ionized calcium concentration to at least 1 mmol/L. In some embodiments, the amount of elemental calcium in the dose to be co-administered with Amiloride and/or an Amiloride derivative can range from about 100 mg per dose to about 2000 mg per dose, more preferably from about 500 mg per dose to about 1000 mg per dose. A preferred oral dosage form such as tablets, capsules or effervescent powders can contain a calcium increasing agent providing about 10 to about 2000 mg of elemental calcium, preferably from about 100 mg to about 1500 mg of elemental calcium, more preferably from about 500 mg to about 1000 mg of elemental calcium.

For parenteral administration, the Amiloride component i.e. Amiloride and/or an Amiloride derivative can be employed in an amount ranging from about 0.005 mg/kg to about 10 mg/kg and preferably from about 0.005 mg/kg to about 2 mg/kg.

In some embodiments, a pharmaceutical composition comprising Amiloride and/or an Amiloride derivative can be typically administered at an initial acceptable dose, and the dose can be escalated up as the subject tolerates it. For example, for administration to a human adult, a test dose of Amiloride and/or an Amiloride derivative is 5 mg/day, which can then be increased up to 10-50 mg/day, once a day, as the patient tolerates it. As used herein if Amiloride and/or an Amiloride derivative is used then the combined amount of each will total 5 mg/day or can be increased to 10-50 mg/day. Tolerance level can be estimated by determining whether stabilization of CHD symptoms is accompanied by improvement or regression of the electrocardiographic alterations. If indicated, the dose of Amiloride and an Amiloride derivative can be increased up to 75 mg BID or TID.

The use of the compositions of the present disclosure represents a novel mechanism of action of Amiloride, with a broader therapeutic approach for CVD subjects, particularly, CHD, hypertension, diabetes mellitus, and left ventricular hypertrophy. Therefore, for additional guidance regarding formulation and dose of Amiloride is known to those skilled in the art. The dosages should be administered and reviewed with special care, which includes monitoring the subject's blood chemistry and ECG patterns. For example, hyperkalemia often attributed to Amiloride was never observed in the subjects participating in the Amiloride trials described herein, despite the absence of hyperkalemia in subjects administered with intravenous doses of Amiloride (1 mg/Kg), in subjects with chronic kidney disease. (Levy Y. et al. Amiloride-sensitive and Amiloride-insensitive kaliuresis in advanced chronic kidney disease. J Nephrol. 2008; 21(1):93-98. In contrast, hyperkalemia is a common secondary effect of Amiloride, as disclosed in various medical textbooks: Berkow et al. (1997) The Merck Manual of Medical Information, Home ed. Merck Research Laboratories, Whitehouse Station, N.J.; Goodman et al. (1996) Goodman & Gilman's the Pharmacological Basis of Therapeutics, 9th ed. McGraw-Hill Health Professions Division, New York; Ebadi (1998) CRC Desk Reference of Clinical Pharmacology. CRC Press, Boca Raton, Fla.; Katzung (2001) Basic & Clinical Pharmacology, 8th ed. Lange Medical Books/McGraw-Hill Medical Pub. Division, New York; Remington et al. (1975) Remington's Pharmaceutical Sciences, 15th ed. Mack Pub. Co., Easton, Pa.; and Speight et al. (1997) Avery's. Drug Treatment: A Guide to the Properties, Choice, Therapeutic Use and Economic Value of Drugs in Disease Management, 4th ed. Adis International, Auckland, Philadelphia.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. These Example embodiments are exemplified through the use of standard cardiology practices of the inventor. In light of the present disclosure and the general level of skill in the art, those of skill will appreciate that the following examples are intended to be exemplary only and that numerous changes, modifications and alterations can be employed without departing from the spirit and scope of the disclosure.

### EXAMPLE 1

### Composition for Oral Administration

The following formulations and manufacturing procedures can be used for the manufacture of tablets containing Amiloride and/or an Amiloride derivative. It should be understood that one skilled in this art will recognize equivalent formulations, which are intended to be included with the scope of this disclosure. A preferable composition tablet comprises the following components:

| Agent | Range (mg) | Amount |
|---|---|---|
| Amiloride and/or Amiloride derivative | 0.05-100 | 5 mg |
| Lactose | 20-30 | 30 mg |
| Starch | 10-150 | 51 mg |
| Magnesium Stearate | 20-50 | 4 mg |
| Gelatin Powder | 50-100 | 20 mg |
| Sugar | 20-120 | 35 mg |

The active agent Amiloride and/or an Amiloride derivative is combined with the other excipients and compressed into tablets using a suitable size tooling. Cure the tablets, cool and package in aluminum foil.

### EXAMPLE 2

### Composition for Oral Administration

The following formulations and manufacturing procedures can be used for manufacture of effervescent tablets containing Amiloride and/or an Amiloride derivative and a calcium-releasing agent. It should be understood that one skilled in this art will recognize equivalent formulations, which are intended to be included with the scope of this disclosure. A second oral composition tablet comprises an effervescent form having the following components:

| Agent | Range (mg) | Amount | |
|---|---|---|---|
| Amiloride and/or Amiloride derivative | 0.05-100 | 5 | mg |
| calcium glubionate and | 500-2000 | 1000 | mg |
| calcium lactobionate | | | |
| (Calcium Sandoz) | | | |
| Citric acid, anhydrous | 600-700 | 650 | mg |
| (granular) | | | |
| calcium bicarbonate | 300-500 | 367 | mg |
| (granular) | | | |
| Sodium carbonate, anhydrous | 20-60 | 40 | mg |
| Flavoring agent (optional) | 10-50 | 25 | mg |
| Colorant (optional) | 0-10 | 5 | mg |
| Sodium benzoate | 5-15 | 7.5 | mg |
| Water | 0-5 | 2 | mg |

Premix sodium benzoate with sodium bicarbonate and Amiloride. Mix color with sodium carbonate. Place citric acid in a bowl of a suitable blender. Add the 2 mg water to the citric acid slowly and mix thoroughly to form a moist blend. Add to the blend, in sequence, while mixing, the calcium glubionate, the sodium bicarbonate mix, and the sodium carbonate-color mix until uniformly distributed. Compress tablets using suitable size tooling. Cure the tablets, cool and package in aluminum foil. Alternatively, the above formulation can be formed into a powder to be admixed with an appropriate amount of water prior to administration.

### EXAMPLE 3

### Composition for Oral Administration

The following formulations and manufacturing procedures can be used for manufacture of non-effervescent tablets containing Amiloride and/or an Amiloride derivative and a calcium-releasing agent (e.g. IDEOS or Calcium Carbonate), It should be understood that one skilled in this art will recognize equivalent formulations, which are intended to be included with the scope of this disclosure.

### EXAMPLE 4

### Composition for Oral Administration

The following formulations and manufacturing procedures can be used for manufacture of effervescent tablets containing Amiloride and two calcium-releasing agents. It should be understood that one skilled in this art will recognize equivalent formulations, which are intended to be included with the scope of this disclosure. A third oral composition tablet comprises an effervescent form having the following components:

| Agent | Range (mg) | Amount | |
|---|---|---|---|
| Amiloride and/or Amiloride derivative | 1-50 | 5 | mg |
| calcium glubionate and | 500-2000 | 1000 | mg |
| calcium lactobionate | | | |
| (Calcium Sandoz) | | | |
| Citric acid, anhydrous | 600-700 | 650 | mg |
| (granular) | | | |
| calcium bicarbonate | 300-500 | 367 | mg |
| (granular) | | | |
| Sodium carbonate, anhydrous | 20-60 | 40 | mg |
| Flavoring agent (optional) | 10-50 | 25 | mg |
| Colorant (optional) | 0-10 | 5 | mg |
| Sodium benzoate | 5-15 | 7.5 | mg |
| Water | 0-5 | 2 | mg |

Premix sodium benzoate with sodium bicarbonate and Amiloride. Mix color with sodium carbonate. Place citric acid in a bowl of a suitable blender. Add the 2 mg water to the citric acid slowly and mix thoroughly to form a moist blend. Add to the blend, while mixing, the calcium glubionate, the sodium bicarbonate mix, and the sodium carbonate-color mix until uniformly distributed. Compress tablets using suitable size tooling. Cure the tablets, cool and package in aluminum foil. Alternatively, the above formulation can be formed into a powder to be admixed with an appropriate amount of water prior to administration.

### EXAMPLE 5

### Composition for Oral Administration

The following formulations and manufacturing procedures can be used for manufacture of effervescent tablets containing Amiloride and two calcium-releasing agents. A fourth oral composition tablet comprises an effervescent form having the following components:

| Agent | Range (mg) | Amount |
|---|---|---|
| Amiloride and/or Amiloride derivative | 1-20 | 5 mg or 10 mg |
| Citric acid, anhydrous | 450-650 | 590 mg |
| (granular) | | |
| Sodium bicarbonate | 750-1000 | 850 mg |
| (granular) | | |
| Sodium bicarbonate, powder | 50-150 | 87 mg |
| Citrus flavor (optional) | 0-50 | 25 mg |
| Water | 0-25 | 15 mg |

The tablet was made by adding Amiloride, citric acid and citrus flavor in a suitable blender. Quickly add the granular sodium bicarbonate and water and mix until a workable mass is formed. Granulate through a suitable screen using a granulator. Spread evenly on a paper-lined tray or a fluid bed dryer. Place a dried granulation in a suitable blender and add powder sodium bicarbonate. Mix well. Compress tablets using a suitable flat face beveled edge tooling. Package in aluminum foil or aluminum tubes.

### EXAMPLE 6

### Single-Blind Trial of Amiloride in Coronary Heart Disease Subjects During 6-Month Follow-Up

This study presents the first evidence documenting that the rapid reversion of angina, symptoms of heart failure, and progressive improvement of the ECG alterations in our subjects receiving Amiloride uncover a novel critical role of RBC in the management and treatment of subjects with CHD.

### Material and Methods

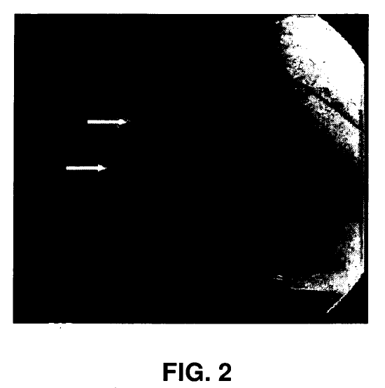

Subjects: 75 consecutive subjects with proved CHD (previous myocardial infarction, 2-3 vessels disease in coronary angiography, as in FIG. 2, or a combined ECG/ECHO ischemic changes) entered this Single-Blind prospective trial of Amiloride. Subjects were evaluated by clinical angina (CCS angina Class II-IV), dyspnea, or arrhythmia. There were 40 hypertensive subjects (aged 53±7; BP 167±13/103±7 mmHg; HR 69±8 l/min) and 35 normotensives (aged 61±7; BP 132±6/75±4 mmHg, HR 61±7 l/min) from both sexes.

Inclusion Criteria: Proved CHD as above, angina (II-IV) and ECG alteration of myocardial ischemia, despite treatment with nitrates, aspirin, calcium-antagonists, statins, and anti-aggregants, while control of BP in hypertensives was achieved by the use of Thiazide diuretics, Angiotensin converting enzyme inhibitor (ACEI), Spironolactone, Angiotensin 1 (AT1) receptor blocker. Exclusion Criteria: Evidence of acute coronary syndrome, AMI, stroke or cardiac arrhythmias; NYHA class III-IV of heart failure, hyperkalemia. Informed consent was obtained in all. Other non CHD causes of chest pain, dyspnea, and arrhythmia had been ruled out.

Trial design: Single-Blind prospective trial of Amiloride (5 mg/daily) in subjects with CHD having decreased RBC K content (≦90.2 mmol/L of cells). In addition to that, a single dose of calcium gluconolactate (1 g/daily) was given to all subjects having plasma ionized ICa²⁺ ≦1.1 mmol/L, while other Laboratory correction and drug assessment were conducted across the first 3-month period (Table 1) Safety control (ECG, Ion Transport Laboratory, BIA, Non-Invasive Hemodynamic) was obtained at 1, 3 and 6 months and 1 year. Clinical control was obtained weekly in the first month or as needed for physician or patient symptoms; thereafter a 3, 6 and 12 months.

### Drug and Laboratory Design

The trial design consisted of administration of low doses of Amiloride (5 mg) daily to each of the treated subjects in the trial within the first 24-h after inclusion with the requirement that all baseline clinical, ECG and laboratory test were completed. The subjects were taken off any drug that might impair RBC transport, such as digitalis, β-blockers, or NSAID's, in subjects with RBC K⁺ ≦90.2 mmol/L cell. Oral calcium gluconolactate (1 g/daily) was administered in all subjects with plasma ionized calcium ≦1.1 mmol/L. Subjects had correction of other plasma electrolyte abnormalities. The subjects were taken off any drug that may affect ventricular repolarization, as digoxin, calcium channel blockers (CCB), diuretics, β-blockers, or the like. Amiloride was discontinued in subjects with persistent angina or evidence of instable episodes after the first 2 weeks of trial. Nitrates and/or CCB were reduced in subjects free of angina after the 2nd week of the Amiloride trial. Anti-aggregants or aspirin in subjects free of angina were discontinued after the 3rd month of treatment.

### Laboratory, ECG, Hemodynamic, and BIA Evaluation.

All subjects underwent: a) Serial ECG for LVH by Cornell or Sokolow-Lyon Criteria (QRS duration times Cornell voltage: R aVL+SV3, with 6 mm [added in women >2440 mm×ms], or Sokolow-Lyon voltage: [SV1+RV5/V6] >38 mm; along with LV strain pattern (classical ST-segment depressed, with inverted T-waves), or LVH voltage criteria with mild LV strain pattern (isoelectric, biphasic or non-symmetrical negative T-waves, without ST-segment depression), and ECG criteria for infarction and/or ischemia (Q-waves from previous AMI, decreased R-wave height, non-progressive R-wave voltages in precordial leads, ST-segment elevation ≧0.1 μV often referred as injury current, ST-segment depression ≧0.1 μV often downsloping from the J point, rectification of the ST segment, and isoelectric, biphasic, negative or inverted T-wave), intraventricular conduction disturbance, and arrhythmias); b) Non-invasive hemodynamic study (DP200M) for cardiac parameters (LV Ejection Time (LVET), LV dP/dT (LV delta pressure-time), Cardiac output and index (CO, Cl), Stroke Volume (SV), Pulse Wave Analysis (PWA) of Central Aortic Blood Pressure (BP), Aortic Systolic (SBP), Diastolic (DBP) and mean (m) BP, Aortic augmentation index; inflection point and reflected wave analysis, Systemic Vascular Resistance and Compliance (SVR/SVC), and Brachial Artery Distensibility (BAD), Brachial Artery Compliance (BAC); c) Ion Transport Studies for RBC (K⁺, Na⁺) plasma (K⁺, Na⁺, Cl⁻, ICa²⁺, Mg²⁺), and 12-hours night urinary volume (K⁺, Na⁺, Cl⁻, pH), d) Bio-Impedance Analysis (BIA, Quantum X, RJL Systems, MI, USA) for Total body Potassium, Total body water/Extracellular Water, Fat Mass (FM) and Fat-Free Mass (FFM) and e) Echocardiography was obtained at entry, 6 and 12 months periods.

### Results

At the 1st Month: Within the 1st month of treatment, all subjects were free of angina and symptoms of heart failure, requiring no sublingual nitrates, calcium-antagonists or β blocker drugs. Plasma K remained normal (range 3.5-5.0 mmol/L), while RBC K increased (84.5±4 vs 93.5±4 mmol/L cell), (p<0.001), and plasma ICa²⁺ was equal or greater than 1.05 mmol/L. The urinary Na⁺/K⁺ rate excretion decreased, while BIA showed significantly decreased Total Body Water and FFM (Fat-Free-Mass).

In hypertensives, LV dP/dT was reduced from 1757±313 vs 1302±276 mmHg/s, (p<0.001), as well as SV Resistance 2114±348 vs 1668±432 dynes/s/cm², (p<0.001), while SVC, and BA Compliance were increased 0.92±0.16 vs 1.22±0.21 ml/mmHg, (p<0.003), and 0.57±0.021 vs 0.069±0.02 ml/mmHg, (p<0.005); but such improvement was unrelated to BP levels.

At the 3rd Month: ST-segment depression or elevation, and isoelectric, biphasic or inverted T-wave improved in most subjects (74%) with CHD despite no nitrates, lower anti-aggregants drugs or aspirin, while in hypertensives LVH voltage criteria was significantly decreased (p<0.001) and LV Strain pattern were improved or almost reversed. Also found within the 1^{st} month, plasma electrolytes were maintained normally, while RBC K⁺ remained improved.

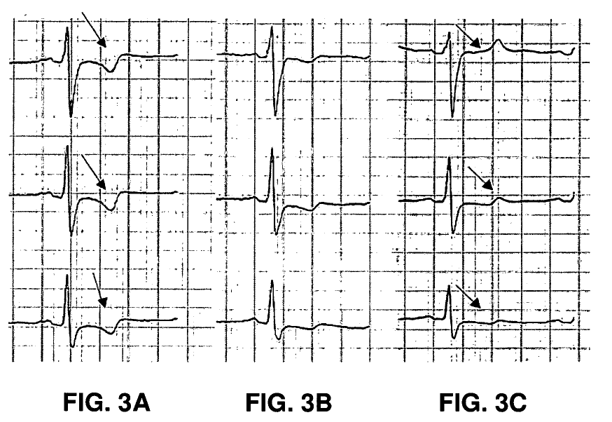

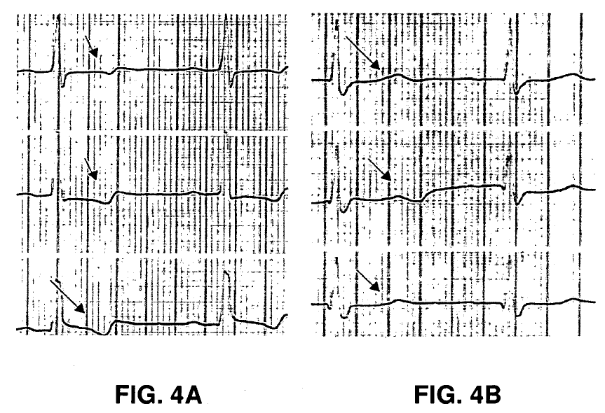

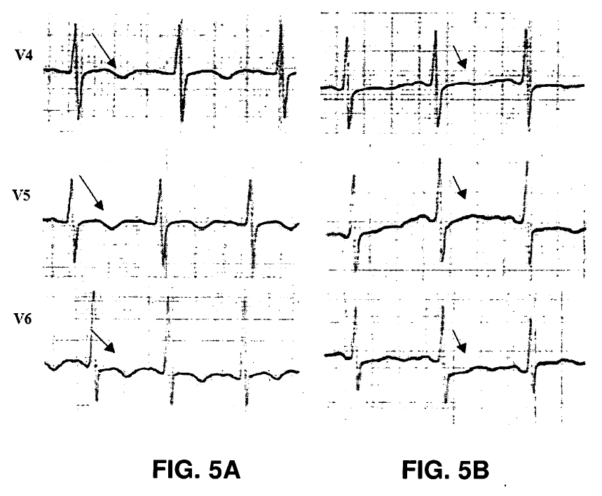

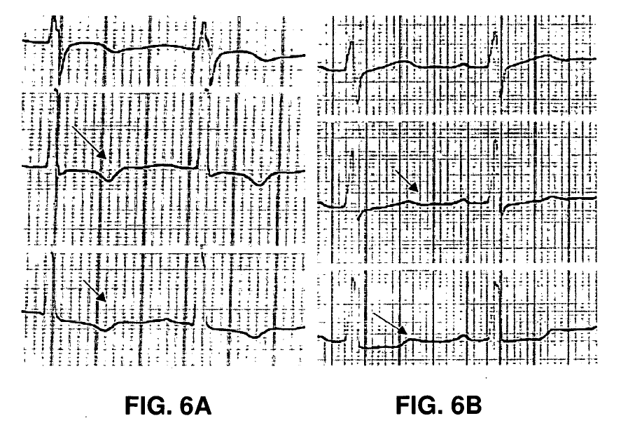

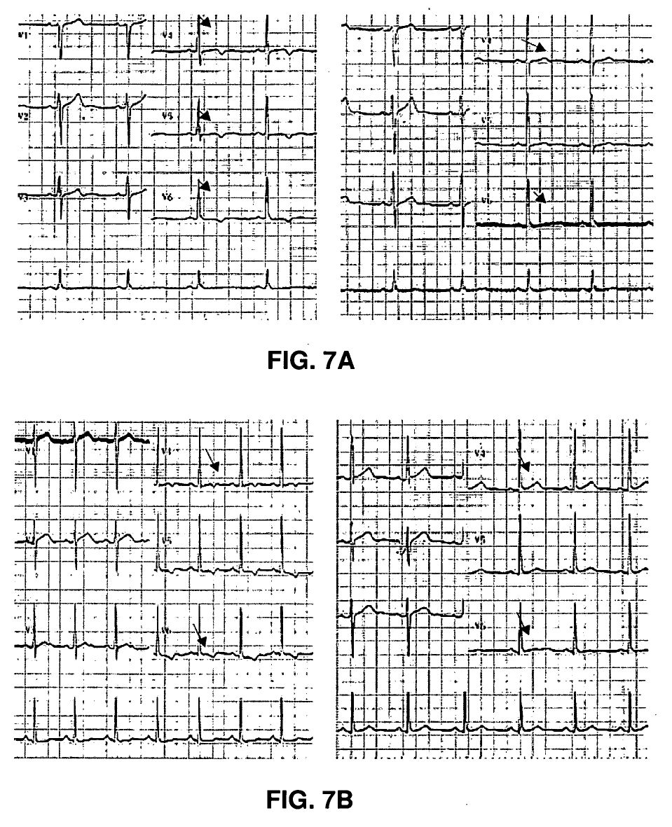

At the 6th Month: The ECG-LVH voltages were significantly reduced in both Cornell 2577±509 mm×ms to 2353±254, (p<0.02) and Sokolow-Lyon criteria 29.3±5.2 mm to 25.1±3.2 (p=0.001), whereas LV Strain pattern had been improved in about 4 out of 5 hypertensives subjects (80%), and QTc interval was prolonged. In ECG-CHD, ST-segment depression or elevation, and inverted T-waves were marked improved in 3 out 4 of normotensive subjects (75%), while others T-waves remained with low voltage or isoelectric. Only (15%) of the subjects having had a previous inferior infarction, and were treated with Amiloride had ECGs that remained unchanged for Q-waves, but these subjects were free of angina. No patient experienced cardiovascular events or death (0%) in the 2 years of follow-up shown in FIGS. 3-7.

### Conclusions

This study presents the first evidence documenting a critical role of RBC in the therapeutic approach and management of CHD. This approach based on the specialized function of RBC in tissue H⁺/K⁺ coupled O₂/CO₂ exchanges (Bohr Effect), and NO-generation strongly support the involvement of RBC in myocardial microcirculation and oxygen unloading in subjects with CHD. The results of this trial with the drug Amiloride provides the first therapeutic approach directed to improved RBC function that likely explains the rapid reversion of the ECG alterations in subjects with myocardial infarction, in subjects with LVH and hypertension, as well as in normotensive diabetics with proved CHD.

### EXAMPLE 7

### Single-Blind Trial of Amiloride in Coronary Heart Disease During 2-Year Follow-Up

### Subjects and Methods

Fifty two (n=52) subjects gave a written consent for this Single-Blind Prospective Long-Term (2-year) Trial of Amiloride in CHD. Subjects were evaluated by clinical (CCS angina Class, dyspnea, and arrhythmia). Other non CHD causes of chest pain, dyspnea, and arrhythmia had been ruled out. Safety control were serial ECG, non-invasive hemodynamics (DynaPulse 200A, CA, USA), Ion Transport Study (plasma and RBC electrolytes, 12-hour urine volume and electrolyte excretion, and plasma and urine creatinine and osmolality), BIA for Body Composition Analysis and estimation of Total Body K⁺ (Quantum X, RJL Systems), and Clinical Biochemistry (FPG, Lipids, Uric acid), were measured at 1, 3, 6, 12, 18, 24 months follow-up period.

### Drug and Laboratory Design

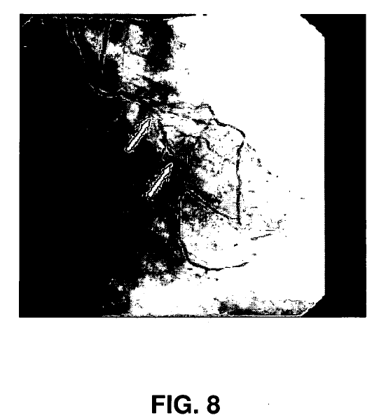

Trial design: Single-Blind prospective Long-term (2-year) trial of Amiloride (5 mg/daily) in subjects with CHD having decreased RBC K⁺ content (≦90.2 mmol/L of cell), while nitrates, aspirin, calcium-antagonists, statins, and anti-aggregants, or other anti-anginal were continued. These subjects were prospectively evaluated in order to assess the effects of Amiloride on reversion of angina and ECG alterations of ischemia in CHD. All of the subjects enrolled had angina, exertional dyspnea, and palpitations despite optimal anti-anginal treatment (nitrates, β-blockers, CCB, aspirin; see Table 2), for more than 6 months before enrollment to this trial. See FIG. 8, a subject in the trial having multiple coronary disease. After written consent, each patient entered this single-blind trial directed to reverse or improve angina and other symptoms of CHD, as well as the ECG alterations of ischemia, and if possible in long-term assay, on improved LV motion/contractility in the echocardiography studies.

In each subject, Amiloride (5 to 10 mg/day) was given before breakfast, while other medication for angina, and associated diseases as hypertension or diabetes, were continued. In addition, subjects with plasma ionized calcium (≦1.0 mmol/L) received 1 g of Ca-gluconolactate until the ionized calcium level was ≧1.0 mmol/L. Following the first 3-month trial, subjects with reversion of angina and improvement of ECG abnormalities of ischemia were asked to continue low doses of Amiloride (5 to 7.5 mg), while nitrates, β-blockers or calcium channel blockers were discontinued, and withdrawn, if no evidence of angina occurred, and if no new no ECG ST-T alteration of ischemia developed. Thus, Amiloride with/without aspirin, and medication for hypertension or diabetes, was the established treatment during the remainder 6 to 12-months. In the last 12-month period, subjects free of angina on Amiloride were evaluated every 6-months. In this trial, control of hypertension was achieved by use of Thiazide, ACEI, Spironolactone, AT1 receptor blocker; in diabetics, a standard treatment (diet, Sulfonylurea, Biguanides, Acarbose, or insulin) was followed in order to achieve a better control of fasting plasma glucose, insulin, and HbA1 levels.

### ECG, Hemodynamic, Laboratory, and BIA Evaluation.

All cases had: a) Serial ECG for LVH by Cornell or Sokolow-Lyon Criteria (QRS duration times Cornell voltage: [R aVL+ SV3, with 6 mm added in women, >2440 mm×ms], Sokolow-Lyon voltage: [SV1+RV5/V6]> 38 mm; along with LV strain pattern [classical ST-segment depressed, with inverted T-waves], or LVH voltage criteria with mild LV strain pattern (isoelectric, biphasic or non-symmetrical negative T-waves, without ST-segment depression), and ECG criteria for infarction and/or ischemia (Q-waves from previous AMI, decreased R-wave height, non-progressive R-wave voltages in precordial leads, ST-segment elevation ≧0.1 μV often referred as injury current, ST-segment depression ≧˜0.1 μV often downsloping from the J point, rectification of the ST segment, and isoelectric, biphasic, negative or inverted T-waves, intraventricular conduction disturbance, and arrhythmias); b) Non-invasive hemodynamic study (DP200M) for cardiac parameters (LV Ejection Time [LVET], LV dP/dT (LV delta pressure-time), Cardiac output and index (CO, Cl), Stroke Volume (SV), Pulse Wave Analysis (PWA) of Central Aortic Blood Pressure [BP], Aortic Systolic (SBP), Diastolic (DBP) and mean (m) BP, Aortic augmentation index; inflection point and reflected wave analysis, Systemic Vascular Resistance and Compliance (SVR/SVC), and Brachial Artery Distensibility (BAD), Brachial Artery Compliance (BAC); c) Ion Transport Studies for RBC (K⁺, Na⁺) plasma (K⁺, Na⁺, Cl⁻, ICa²⁺, Mg²⁺), and 12-hours night urinary volume (K⁺, Na⁺, Cl⁻, pH), d) Bio-Impedance Analysis (BIA, Quantum X, RJL Systems, MI, USA) for Total body Potassium, Total body water/Extracellular Water, Fat Mass (FM) and Fat-Free Mass (FFM) and e) Echocardiography was obtained at entry, 6 and 12, 24 months periods.

### Statistical Analysis

The statistical analysis was performed using the Student's t-test and analysis of variance. To reduce the probability of significant differences arising by chance, Bonferroni's correction was applied following analysis of variance. All measurement are presented as the mean±S.D., and differences were considered significant when p<0.05.

### Results

Table 2, presents the clinical data of the 52 subjects studied, including CCS Angina Class (Canadian Cardiovascular Society Class), associated symptoms, associated disease, and treatment received at the time of enrollment.

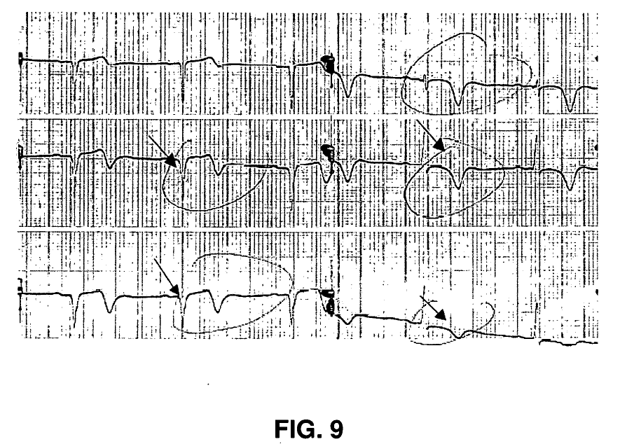

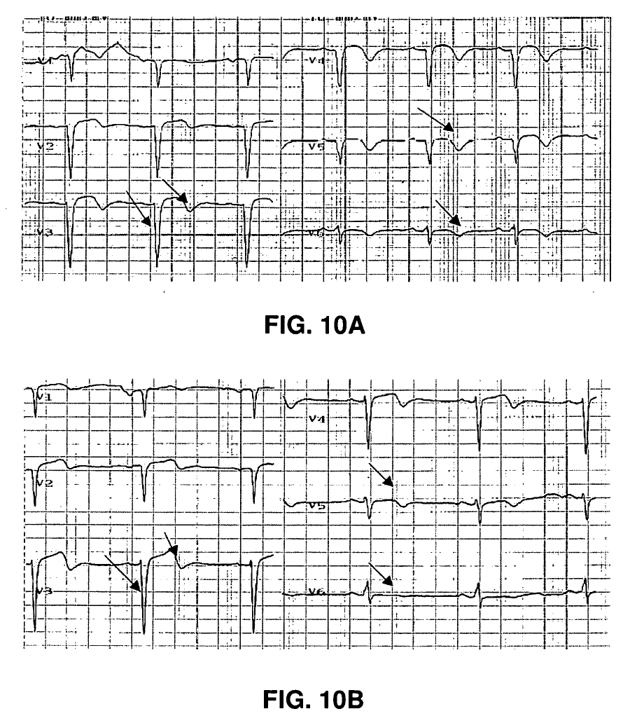

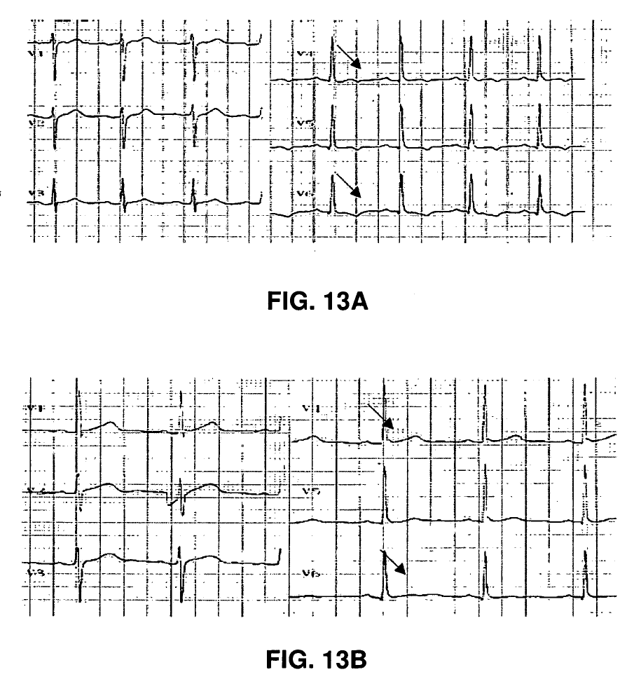

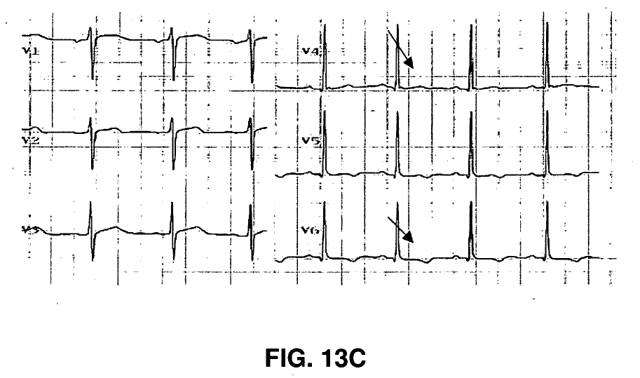

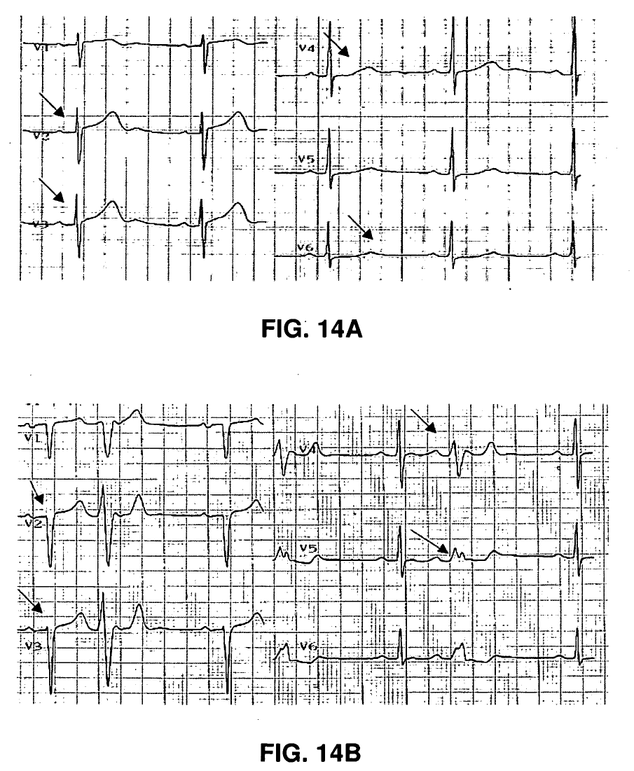

A graphic description is presented of the severity of the coronary artery obstruction by angiography (See FIG. 1.), ECG evidence of previous AMI in these subjects (See FIG. 9, ECG example of myocardial necrosis and ischemia in precordial leads V1-V3 (left) and V4-V6 (right)), ECG changes improved in subjects during Amiloride Trial (See FIGS. 10A-12C), and ECG ischemic recurrence after omission of Amiloride (See FIGS. 13C and 14B).

Table 3, shows the clinical, ECG and the echocardiographic findings after completion of 2-year Amiloride trial.

Other important studies, including Ion Transport (Defective Red-Blood-Cell Potassium content and transport), Biochemistry Laboratory (FPG, Lipids, Uric Acid), Non-Invasive Hemodynamic (Cardiovascular parameters, arterial pulse waveform analysis), BIA (TBK, Body Water Content and distribution, Fat Mass) has been separated for further discussion.

FIG. 10A illustrates an ECG of an elderly male subject having had with extensive AMI, emergency angioplasty, and LAD stent (2006), with sustained ECG changes after 1-year episode of AMI (2007). (FIG. 10B) illustrates an ECG showing unchanged ECG features (arrows) despite optimal medical treatment, but no angina, Duke treadmill Score of −1. This subject did not participate in the exemplary trial.

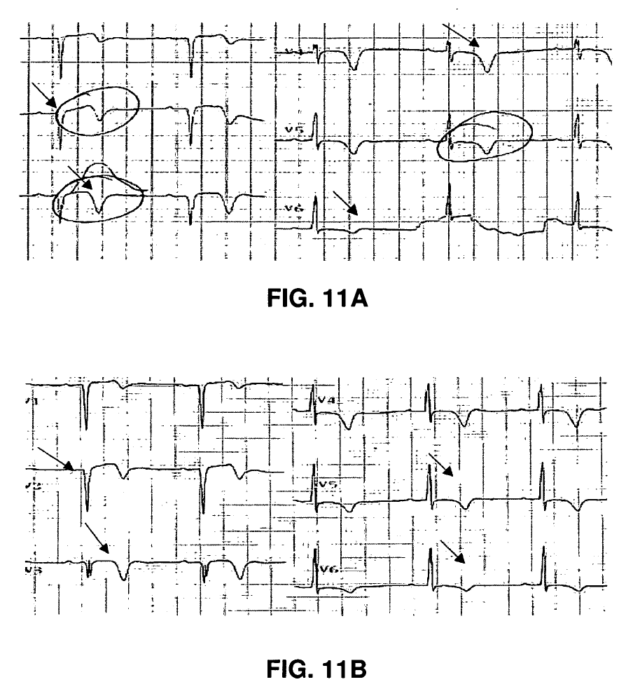

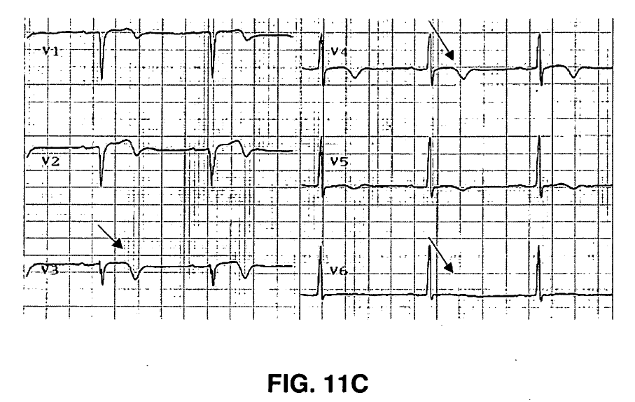

FIGS. 11A-11C depicts ECG recordings of an elderly male with previous extensive myocardial infarction, angina and heart failure. In FIG. 11A, the subject presents with an ECG after previous extensive, angina and heart failure. Arrows are shown to depict the ECG alterations indicating signs of cardiovascular disease 1-year later. In FIG. 11B, the subject presented with an unchanged ECG during a 3-year follow-up, despite optimal medical treatment. The subject continued with angina and had a Duke Treadmill Score of −3, before entering the Amiloride trial (January 2008). In FIG. 11C, the ECG tracing shows rapid reversion of ST-T alterations (V2-V6) after 2-month Amiloride trial (5 mg), and optimal medical treatment. The patient was free of angina or symptoms of heart failure, with DUKE TREADMILL SCORE=+4.3 (2008).

TABLE 2
Baseline Clinical and CHD presentation in Single-Blind
Amiloride Trial

| Cases | Number | Percentage (%) |
|---|---|---|
| Female/Male | 15/37 | 28.8/72 |
| Age (years) | 63.1 ± 6.6 | 100 |
| Angina Presentation Class | | |
| Refractory Angina (II-III) | 25 | 48.2 |
| Unstable angina (II-III) | 9 | 17.2 |
| Stable Angina (II-III) | 18 | 34.4 |
| Associated Symptoms | | |
| Palpitation | 30 | 58 |
| Dyspnea | 13 | 25 |
| Heart Failure | 9 | 17 |
| Associate Diseases | | |
| Previous AMI | 34 | 65.5 |
| Hypertension | 37 | 72.4 |
| Diabetes type 2 | 23 | 44.8 |
| Drug Treatment at entry | | |
| Aspirin | 52 | 100 |
| Nitrates | 52 | 100 |
| Statins | 52 | 100 |
| Clopidogrel | 32 | 62 |
| B-Blocker | 21 | 40.3 |
| CCB | 23 | 44.2 |
| ACEI-Spironolactone | 27 | 52 |
| Glibenclamide | 23 | 44.8 |

TABLE 3
Clinical Data, ECG and Echocardiographic Changes Following
Completion of 2-year Amiloride Trial

| Clinical and Study Changes | Number | Percentage (%) |
|---|---|---|
| Subjects with Angina | 52 | 100 |
| Days for Improvement | 6.2 ± 3.5 | 86.5 |
| Reversion of Angina | 43 | 82.7 |
| Free of Angina (1-2 years) | 37 | 71.2 |
| New CVD Events | | |
| None | 41 | 79 |
| ACS | 7 | 13.5 |
| Ventricular Arrhythmias | 5 | 9.6 |
| AMI/Stroke/Death | 0 | 0 |
| EGG Alterations | | |
| Reverted to Normal | 18 | 34.5 |
| Improved Changes | 23 | 44.2 |
| Weeks for Improvement | 3.4 ± 1.5 | 61 |
| Unchanged/Worst | 10 | 19.2 |
| Echocardiographic Changes | | |
| Normal Contractility | 18 | 34.5 |
| Altered Hypokinesia | 34 | 65.5 |
| Reversed Hypokinesia | 27 | 52 |
| Unchanged Hypokinesia | 7 | 13.5 |

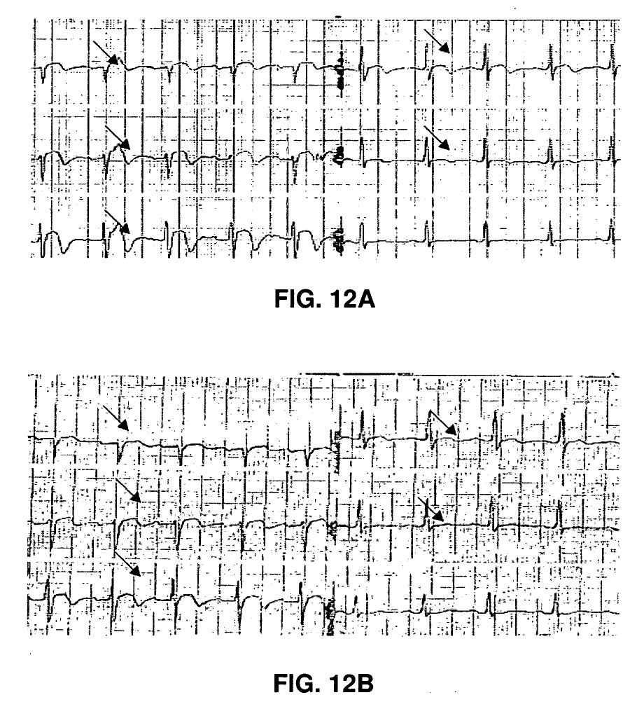

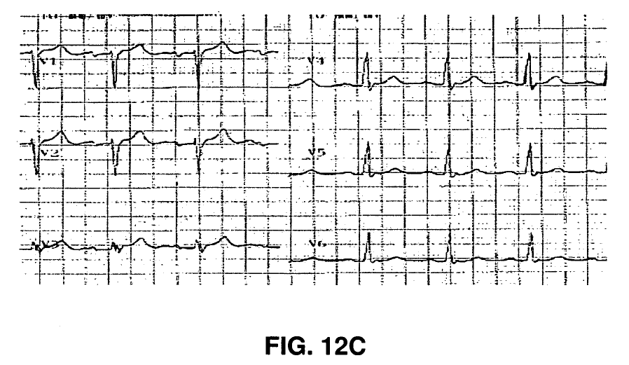

FIGS. 12A-12C further illustrate the effectiveness of the pharmaceutical composition in completely normalizing alterations in the ECG results. Specifically, it was found that significant ECG improvement of ST-segment, and T-waves (V1-V6) with amiloride, calcium and statins only. FIG. 12C reveals that the subject having had an AMI, was able to return to a normal ECG tracing after 1½ years of treatment. The subject was thereafter asymptomatic, receiving only Amiloride, calcium, and statins. The subject's Duke treadmill Scores improved to and 6.23. Normal LV contractility and function in serial echocardiograms is shown for the subject in FIG. 12C. As shown in FIGS. 13A-13C and 14A and 14B, ECG recordings of subjects, who showed improvement in ischemic changes during the Amiloride trial but then relapsed, as shown in FIGS. 13C and 14B after discontinuing treatment with Amiloride but while still taking conventional medicaments for angina and diabetes.

### Conclusions

This study presents the first evidence showing a rapid reversion or improvement of angina, ECG and echocardiographic studies on subjects receiving Amiloride during a 2-year single-blind trial. The pharmacological and therapeutic efficacy of Amiloride appears to target the specialized functions of RBC in tissue H⁺/K⁺ coupled O₂/CO₂ transport, vascular rheology and blood cell aggregation, strongly supporting a critical role of red blood cell (RBC) in the regulatory mechanism of myocardial microcirculation and oxygenation in subjects with CHD. It is believed that Amiloride rapidly enhances cell K⁺ uptake, and particularly RBC K⁺ transport at the capillary beds, and at distal tubular cell of kidney during intravenous furosemide administration. (Delgado-Almeida A. Assessing Cell K Physiology in Hypertensive Subjects: A New Clinical and Methodological Approach. Am J Hypertens, (2006), 19: 432-436).

Although this was not a randomized double-blind placebo-controlled study of Amiloride, the fact that most subjects had refractory angina and unstable angina (65.5%), despite optimal medical treatment, at the time of enrollment, made it possible to assume that each patient serves as it s own control without Amiloride.

Further, the rapid reversion of ECG changes of myocardial ischemia, sustained through the 2-year investigation, as well the recurrence of angina and ECG alterations in the subjects after withdrawal of Amiloride, strongly support a specific role of Amiloride in such mechanisms including oxygen delivery in myocardial ischemia. In fact, a long-term assay from our laboratory has shown that Amiloride reverses a hereditable defect in cell K uptake by RBC that may critically impair the tissue H⁺/K⁺ coupled O₂/CO₂ exchanges at the myocardium (results not shown).

While not wishing to be bound by any particular theory, it is believed that the nitrate effects on subjects with CHD may be strongly correlated with conformation changes of reduced-hemoglobin, H⁺ uptake, and NO-generation within the RBC. Such a theory may suggest a mechanism of H⁺/K⁺ exchange at capillary beds which needs to be preserved or enhanced in subjects with CHD. It should noted that K⁺ is the major buffer base available in RBC, and as consequence disturbance in RBC K⁺ content and uptake may impair the role of hemoglobin in tissue oxygen transport. Finally, the observation that both angina and ECG alterations of ischemia recurred shortly after Amiloride withdrawal further supports a novel therapeutic effect of Amiloride that may be related to every instant where tissue oxygen delivery is impaired or diminished.

### EXAMPLE 7

### Double-Blind Trial Placebo-Controlled Trial of Amiloride of Amiloride on Angina, ECG alterations, and Exercise Tolerance in Coronary Heart Disease

### Subjects and Methods

A double blind placebo-controlled, randomized trial was conducted in 25 subjects with stable chronic angina. Subjects received a daily dose of Amiloride (5 mg) or placebo (5 mg), for 4 weeks. Each subject gave their written consent to participate in the trial, and continued with their regular anti-anginal treatment, as well as drugs for the primary condition as diabetes, CHD or hypertension. All of the subjects were evaluated before and after the double-blind trial, thereby measuring a subject's angina clinical score (CCS Angina Class), ECG, and exercise tolerance as assessed by the Duke Treadmill Score (DTS). Methods for using the DTS with patients in need of coronary intervention and its acceptance as an accurate predictor of cardiac morbidity and mortality in CHD and CAD patients has been well established. See for example Azeem T., et al., Int J. Clin. Pract. (2001), 55(1):10-13. The double blind placebo-controlled, randomized Amiloride study was conducted between (November 2007) and (February 2008). Participants' clinical and cardiac parameters were evaluated either monthly, quarterly, half yearly and yearly. Table 4 presents the demographic characteristics of the studied groups. There were no significant differences between the treated and placebo groups with respect to past or present history of hypertension, diabetes mellitus, myocardial infarction, angina status, or clinical laboratory tests.

TABLE 4
Clinical Characteristics of the Study Groups In The
Double-Blind Placebo-Controlled Trial of Amiloride

| | Amiloride | |
|---|---|---|
| | A | Placebo |
| | (N = 14) | (N = 11) |
| Cases N = Gender (f/m) | 14 (5/9) | 11 (4/7) |
| Age | 62 ± 7 | 64 ± 7 |
| Body Weight (Kg) | 76 ± 9 | 73 ± 7 |
| Body Height (cm) | 168 ± 7 | 163 ± 6 |
| Essential Hypertension | 5 | 3 |
| Diabetes Mellitus Type-2 | 2 | 4 |
| Acute Myocardial Infarction | 6 | 5 |
| Dyspnea | 7 | 5 |
| CCS Angina Type II | 8 | 7 |
| CCS Angina Type III | 6 | 4 |
| Blood Pressure (mm Hg) | 131 ± 14/ | 142 ± 13/ |
| | 79 ± 7 | 83 ± 7 |
| Heart Rate (l/min) | 74 ± 17 | 71 ± 18 |
| T Cholesterol (mg %) | 168 ± 24 | 163 ± 24 |
| HDL (mg %) | 43 ± 7 | 39 ± 10 |
| Red-Cell K (mmol/Lc) | 89 ± 5 | 87 ± 6 |

CCS (Canadian Cardiovascular Society);
(*) Non-Statistical differences between groups.

The results of the double-blind placebo-controlled Amiloride trial is presented in Table 5. Results obtained from the trial and presented in Table 5 surprisingly shows that the subjects treated with Amiloride significantly improved their Duke Treadmill Score even after only one month of therapy, in both Low Risk Score and High Risk Score, compared to subjects receiving placebo. The statistically significant improvement in the DUKE TREADMILL SCORE in these subjects were obtained without any adverse episodes of cardiac arrhythmia, despite similar ETT Time and Double-BP Product. Twelve out of 14 subjects that were treated with Amiloride were also free of angina (71%) and 9 out of 14 had improved ST-T alterations of ischemia (64%), compared with only 2 cases with reduced incidence of angina (18%), and none (0%) with improved ECG in the placebo controlled group.

Table 5, presents the Exercise Treadmill components and Duke Treadmill Score in the double-blind placebo-controlled trial of Amiloride.

TABLE 5
Duke Treadmill Score at 1-Month trial in Subjects with CHD
and Angina Receiving Amiloride or Placebo

| | Amiloride | Placebo | |
|---|---|---|---|
| | (5 mg/d) | (5 mg/d) | P Value |
| Cases (f/m) | 5/9 | 4/7 | 0.37 |
| Age | 62 ± 7.4 | 64 ± 6.7 | 0.49 |
| Basal BP (mm Hg) | 131 ± 14/79 ± 7 | 142 ± 13/82 ± 9 | 0.07 |
| Basal HR (b/min) | 74 ± 17 | 71 ± 18 | 0.63 |
| Peak Exercise BP | 177 ± 19/94 ± 10 | 180 ± 23/99 ± 13 | 0.75 |
| Peak Exercise HR | 144 ± 19 | 137 ± 15 | 0.29 |
| Double-Product | 25700 ± 4633 | 24600 ± 3864 | 0.53 |
| Exercise Time (min) | 7.95 ± 2.8 | 5.3 ± 2.5 | <0.05 |
| Baseline DTS | −2.28 ± 7.0 | −3.44 ± 6.9 | 0.28 |
| Subjects (n/%) | (14/100%) | (11/100%) | |
| 1-Month DTS | 7.90 ± 4.2 | −4.75 ± 4.4 | <0.00000008 |
| | (14/100%) | (11/100%) | |
| Low Risk Value | *8.6 ± 2.3 | *9.0 ± 4.2 | 0.00352 |
| Subjects (n/%) | (9/64.3%) | (2/18%) | |
| High Risk Value | *−3 ± −2.6 | *−5.9 ± −5.0 | 0.25 |
| Subjects (n/%) | (5/35.7%) | (9/81.8%) | |
| V. Arrhythmia | 0% | 3 (14%) | <0.000001 |

DTS (Duke Treadmill Score);
*= Changes from baseline to 1-month trial of Amiloride and placebo;
V. Arrhythmia (Ventricular arrhythmias).

### Conclusions

The results of the double-blind trial of Amiloride, presented herein further confirms the therapeutic and pharmacological effect of low doses of Amiloride (5-10 mg/day) on the reversion of refractory angina, unstable, or chronic angina (CCS Class II-IV) in subjects with CHD (Macrovascular CHD and Microvascular CHD). The fact that this drug was able to improve the Duke Treadmill Score, while reversing the ST-segment and T-wave alterations of myocardial ischemia, surprisingly supports a novel mechanism of action. Conclusions from the results lead to the hypothesis that pharmaceutical compositions comprising low doses of Amiloride can be used in methods and treatments to improve functional capacity of hemoglobin, and oxygen unloading in capillary beds of the ischemic myocardium found in CHD subjects. The present pharmaceutical compositions and methods of treating CHD subjects with different grades of coronary occlusion, coronary artery endothelial dysfunction, coronary bypass surgery or coronary interventions with pharmaceutical compositions of the present disclosure comprising Amiloride and/or an Amiloride derivative, may suggest a new paradigm in the treatment of CHD in particular, and in cardiovascular disease in general.

What is claimed is:

1. A pharmaceutical composition comprising:
(a) an effective amount of at least one 3,5-diamino-6-chloro-N-(diaminomethylene) pyrazinecarboxamide monohydrochloride, dihydrate derivative comprising pirazinoylguanidine, benzamil, dichlorobenzamil, 5-(N,N-dimethyl)-Amiloride, 5-(N-ethyl-N-isopropyl)-Amiloride, (N,N-hexamethylene)-Amiloride, 5-(N-methyl-N-isobutyl)-Amiloride, and Amiloride citrate;
(b) an effective amount of a calcium increasing agent; and
(c) a pharmaceutically acceptable excipient.

2. The pharmaceutical composition according to claim 1, wherein the 3,5-diamino-6-chloro-N-(diaminomethylene) pyrazinecarboxamide monohydrochloride, dihydrate derivative is Amiloride citrate.

3. The pharmaceutical composition of claim 1, wherein the effective amount of the 3,5-diamino-6-chloro-N-(diaminomethylene) pyrazinecarboxamide monohydrochloride, dihydrate derivative is present in the composition in an amount ranging from about 0.5 mg to about 100 mg.

4. The pharmaceutical composition of claim 1, wherein the effective amount of a calcium increasing agent is an amount that raises the plasma ionized calcium concentration to at least 1.0 mmol/L.

5. A method for treating coronary heart disease in normotensive subjects, the method comprising administering a therapeutically effective composition having at least one of 3,5-diamino-6-chloro-N-(diaminomethylene) pyrazinecarboxamide monohydrochloride, dihydrate, an 3,5-diamino-6-chloro-N-(diaminomethylene) pyrazinecarboxamide monohydrochloride, dihydrate derivative to a normotensive subject in need thereof.

6. The method of claim 5, wherein 3,5-diamino-6-chloro-N-(diaminomethylene) pyrazinecarboxamide monohydrochloride, dihydrate comprises an effective amount of a derivative of 3,5-diamino-6-chloro-N-(diaminomethylene) pyrazinecarboxamide monohydrochloride, dihydrate.

7. The method of claim 5, wherein the administering a therapeutically effective composition further comprises administering a calcium increasing agent when the subject has a plasma ionized calcium level of less than or equal to 1.0 mmol/L.

8. A method for treating coronary vascular disease, the method comprising administering a therapeutically effective composition having at least one of 3,5-diamino-6-chloro-N-(diaminomethylene) pyrazinecarboxamide monohydrochloride, dihydrate and a derivative thereof to a normotensive subject in need thereof wherein the CVD is selected from the group consisting of myocardial infarction, dyspnea, arrhythmia, angina pectoris, hypertension, left ventricular hypertrophy, diabetes, and dyslipidemia.

9. The method of claim 8, wherein the 3,5-diamino-6-chloro-N-(diaminomethylene) pyrazinecarboxamide monohydrochloride, dihydrate or derivative thereof and the calcium increasing agent are administered in the same composition.

10. The method of claim 8, wherein the 3,5-diamino-6-chloro-N-(diaminomethylene) pyrazinecarboxamide monohydrochloride, dihydrate or derivative thereof and the calcium increasing agent are administered as separate compositions.

11. The method of claim 8, wherein the 3,5-diamino-6-chloro-N-(diaminomethylene) pyrazinecarboxamide monohydrochloride, dihydrate or derivative thereof and the calcium increasing agent are administered at the same time.

12. The method of claim 8 wherein the 3,5-diamino-6-chloro-N-(diaminomethylene) pyrazinecarboxamide monohydrochloride, dihydrate or derivative thereof and the calcium increasing agent are administered at different times.

13. The method of claim 8, wherein the administering a therapeutically effective composition comprises administering at least one of 3,5-diamino-6-chloro-N-(diaminomethylene) pyrazinecarboxamide monohydrochloride, dihydrate, a derivative thereof and a calcium increasing agent, each in an amount that substantially improves or reverses at least one clinical symptom related to CVD selected from the group consisting of: improvement or normalization of an ST-segment alteration, T-wave alteration, improvement or normalization of voltage of LVH, and/or LV strain in LVH, improved Duke Treadmill Score, exertional dyspnea, improved LV contractility, a normalization of red blood-cell potassium homeostasis and a reduction in frequency or duration of a ventricular arrhythmias.

14. The method of claim 13, wherein the administering at least one of 3,5-diamino-6-chloro-N-(diaminomethylene) pyrazinecarboxamide monohydrochloride, dihydrate, a derivative thereof and a calcium increasing agent comprises administering Amiloride or Amiloride derivative in an amount ranging from about 0.01 mg/Kg to about 1 mg/Kg.

15. The method of claim 8, wherein the administering the composition comprises administering the composition orally, sublingually, intravenously, intraperitoneally and intramuscularly.

16. A method for treating coronary heart disease in a normotensive subject, the method comprising administering a therapeutically effective-composition having at least one of 3,5-diamino-6-chloro-N-(diaminomethylene) pyrazinecarboxamide monohydrochloride, dihydrate and a derivative thereof.

17. The method according to claim 16, wherein the cardiovascular disease comprises myocardial ischemia, oxidative stress, coronary heart disease, coronary endothelial dysfunction, left ventricular hypertrophy, ventricular arrhythmia, angina, dyspnea, peripheral vascular disease, stroke, hypertension, and diabetes.

18. The method according to claim 16, further comprising administering a calcium increasing agent to the subject if the subject has a plasma ionized calcium level less than 1.0 mmol/L.

19. A method for treating a cardiovascular disease (CVD) in a patient diagnosed as having CVD or at risk for developing CVD, the method comprising administering to a subject in need of such treatment a therapeutically effective amount of at least one of 3,5-diamino-6-chloro-N-(diaminomethylene) pyrazinecarboxamide monohydrochloride, dihydrate and a derivative thereof, wherein the CVD is associated with a dysfunction in red blood cell H⁺/K⁺ transport, the cardiovascular disease comprises myocardial ischemia, oxidative stress, microvascular coronary heart disease, non-obstructive heart disease, coronary endothelial dysfunction, left ventricular hypertrophy, ventricular arrhythmia, angina, dyspnea, peripheral vascular disease, cerebrovascular disease, stroke, hypertension, and diabetes.

20. The method according to claim 20, wherein the therapeutically effective amount of the composition improves or reverses at least one clinical symptom related to CVD selected from the group consisting of improvement or normalization of: an ST-segment alteration, T-wave alteration, LVH, and/or LV strain in LVH voltage, a Duke Treadmill Score, a reduction in the severity or duration of angina pectoris, exertional dyspnea, arrhythmias, improved LV contractility, a normalization of red blood-cell potassium homeostasis and a reduction in frequency or duration of a ventricular arrhythmias when administered to the subject.

21. The method of claim 20, further comprising measuring the plasma ionized calcium concentration of the subject diagnosed as having CVD or at risk for developing CVD and administering a composition comprising 3,5-diamino-6-chloro-N-(diaminomethylene) pyrazinecarboxamide monohydrochloride, dihydrate, a derivative thereof, a calcium increasing agent and a pharmaceutically acceptable excipient, if the subject has a plasma ionized calcium level of less than or equal to 1.0 mmol/L.
